(19) United States
(12) Patent Application Publication
(10) Pub. No.: US 2024/0116939 A1
(43) Pub. Date: Apr. 11, 2024

(54) CHIRAL OR RACEMIC PYRIMIDINE-FUSED DIAZEPINONE DERIVATIVES, PREPARATION METHOD, AND APPLICATION THEREOF

(71) Applicant: SUN YAT-SEN UNIVERSITY, Guangzhou (CN)

(72) Inventors: Liqin Qiu, Guangzhou (CN); Xiaoding Jiang, Guangzhou (CN); Xu Qian, Guangzhou (CN)

(21) Appl. No.: 18/512,177

(22) Filed: Nov. 17, 2023

Related U.S. Application Data
(63) Continuation of application No. PCT/CN2022/092681, filed on May 13, 2022.

(30) Foreign Application Priority Data
May 17, 2021 (CN) 202110534270.0
May 12, 2022 (CN) 202210513147.5

(51) Int. Cl. C07D 487/04; A61P 25/24

(52) U.S. Cl. CPC C07D 487/04; A61P 25/24; C07B 2200/07

(57) ABSTRACT

The present disclosure relates to a technical field of chiral compound synthesis, and particularly to chiral or racemic pyrimidine-fused diazepinone derivatives, preparation method, and application thereof. A structural formula of the chiral or racemic pyrimidine-fused diazepinone derivatives or pharmaceutically acceptable salts thereof is as shown in formula (I),

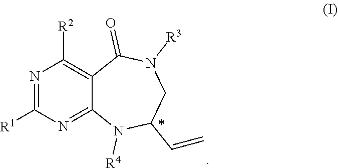

The present disclosure further provides a preparation method of the chiral or racemic pyrimidine-fused diazepinone derivatives, the method includes using pyrimidine allyl compound intermediates as raw materials, using iridium complexes formed by an interaction between iridium compounds and phosphoramidite ligands as catalysts, and obtaining the chiral or racemic pyrimidine-fused diazepinone derivatives by reactions under action of alkalis. The derivatives have good inhibitory effects on GR, cofilin-1, and NF-κB protein, can reverse apoptosis of PC12 cells caused by corticosterone in a depressed state, and can be applied in the preparation of drugs or their lead compounds for preventing or treating depression.

### CROSS-REFERENCE TO RELATED APPLICATIONS

The present application is a continuation application of PCT application No. PCT/CN2022/092681 filed on May 13, 2022. which claims the benefit of Chinese Patent Application No. 202110534270.0 filed on May 17, 2021 and Chinese Patent Application No. 202210513147.5 filed on May 12, 2022. The contents of all of the aforementioned applications are incorporated by reference herein in their entirety.

### FIELD

The subject matter herein generally relates to a technical field of chiral compound synthesis, and particularly to chiral or racemic pyrimidine-fused diazepinone derivatives, preparation method, and application thereof.

### BACKGROUND

A benzodiazepine skeleton is an important structural unit in drug molecules, and its derivatives usually have a wide range of pharmacological activities. With the benzodiazepine skeleton as the key parent nucleus, a series of promising small molecules have been developed for different pathological features. As a structural analog, pyrimidine-fused diazepinone derivatives have received widespread attention from synthetic chemists due to their potential pharmacological activities, and the study on the preparation method of the pyrimidine-fused diazepinone derivatives occupies an extremely important position in the research and development of new drugs. In 1969, Santilli's research group prepared the pyrimidine-fused diazepinone derivatives for the first time, and the preliminary activity screening experiments showed that they have a certain degree of sedative activity and also exhibit antispasmodic effects [Kim, D. H.; Santilli, A. A. J. Med. Chem.1969, 12, 1121-1122.] Subsequently, scientific researchers synthesized a variety of pyrimidine-fused diazepinone derivatives with different structures, and found that they have different biological activities. For example, the compounds provided by Seto's research group in 2005 have a strong inhibitory effect on the neurokinin receptor NK1 (K_{B}=0.480 nM), which could increase an effective bladder capacity of guinea pigs, and show good potential for the treatment of urinary incontinence [Seto, S.; Tanioka, A.; Ikeda, Ni; Izawa, S. Bioorg. Med. Chem. 2005, 13, 5717-5732.]. In 2008, Schulz's research group reported that a kind of pyrimidine-fused diazepinone derivatives can effectively inhibit kinase PDK1, and some of these compounds have particularly outstanding inhibitory effects on kinase PDK1, with the IC₅₀ value reaching a picomolar level [Schulz, M.; Burgdorf, L. T. ; Finsinger, D.; Blaukat, A.; Greiner, H.; Esdar, C.; Kreysch, FL-G.; Henzler, T. PCT Int. Appl. 2008. US 20080318934 A1, 20081225.]. In 2012, Ding's research group designed and synthesized a series of pyrimidine-fused diazepinone derivatives, and found that these derivatives have good in vitro inhibitory effects on EGFR protein and its clinically relevant mutants, with the inhibitory activity of some of these derivatives on EGFR^{L858R/T790M} reaching a nanomolar level (IC₅₀=14 nM); at the same time, this type of derivatives can also inhibit the proliferation of non-small cell lung cancer cells H1975 and HCC827 [Xu, S.; Zhang, L., Chang, S.; Luo, J; Lu, X.; Tu, Z.; Liu, Y.; Zhang, Z.; Xu, Y; Rena, X.; Ding, K. Med. Chem. Commun. 2012,3, 1155-1159]. The pyrimidine-fused diazepinone derivatives designed and synthesized by Cuevas-Cordobes and Almansa-Rosales in 2017 have a strong affinity to the substructure α2δ of the voltage-gated calcium channel (VGCC), especially have strong inhibitory effects on α2δ−1; in addition, this type of derivatives can also effectively inhibit phenylephrine transporters (NET); dual inhibitory effects make this type of derivatives have good application prospects in the treatment of brain injury and related diseases [Cuevas-Cordobes. F.; Almansa-Rosales, C. PCT Int. Appl. 2017, WO 2017191304 A1, 20171109.].

Major depressive disorder (MDD) is a stubborn disease faced by modern medicine, and there is still a lack of effective treatments and drugs. MDD induces nervous system dysfunction, disrupts the functions and structural connections of neural circuits for regulating emotions. Glucocorticoids (GCs) and their receptors (GR) are important signaling molecules for emotion regulating response. Chronic stress and HPA dysregulation promote neuroinflammation, which leads to an increase in pro-inflammatory cytokines and chemokines, such as NF-κB. The inflammation and UPA axis dysfunction induced by MDD would further induce neural synaptic plasticity. The process of neuroplasticity is controlled by regulatory proteins, and cofilin-1 is one of the key regulators, therefore, GR, NT-κB, and cofilin-1 are considered as key targets for the treatment of MDD.

In summary, the pyrimidine-fused diazepinone derivatives, which have structural similarities with benzodiazepines, possess broad biological activities, and have potential research value. Although some progress has been made in their synthesis, the asymmetric catalytic method for constructing such derivatives has not yet been reported. Therefore, the development of effective asymmetric synthesis methods is of great significance for enriching the structures of such derivatives and for drug development.

### SUMMARY

In order to overcome shortcomings and deficiencies of the above-mentioned prior art, a primary purpose of the present disclosure is to provide chiral or racemic pyrimidine-fused diazepinone derivatives or pharmaceutically acceptable salts thereof. The pyrimidine-fused diazepinone derivatives of the present disclosure are levorotatory forms, dextrorotatory forms, or racemic forms. The levorotatory forms are levorotatory pure products or levorotatory excess enantiomeric mixtures. The dextrorotatory forms are dextrorotatory pure products or dextrorotatory excess enantiomeric mixtures. The racemic forms are enantiomeric mixtures, and an ee value of the racemic forms is 0.

Another purpose of the present disclosure is to provide a preparation method of the above-mentioned chiral or racemic pyrimidine-fused diazepinone derivatives. The preparation method of the present disclosure is to use pyrimidine allyl compounds as substrates through catalytic intramolecular allyl amination reaction, to effectively synthesize the pyrimidine-fused diazepinone derivatives and their enantiomers or racemates. The preparation method of the present disclosure can achieve high efficiency and high enantioselectivity to synthesize optically active pyrimidine-fused diazepinone derivatives containing central chirality.

Another purpose of the present disclosure is to provide an application of the above-mentioned chiral or racemic pyrimidine-fused diazepinone derivatives or pharmaceutically acceptable salts thereof in the preparation of drugs or their lead compounds for preventing or treating depression.

The purpose of the present disclosure is achieved through the following solutions:

Chiral or racemic pyrimidine-fused diazepinone derivatives or pharmaceutically acceptable salts thereof, a structural formula of the pyrimidine-fused diazepinone derivatives is as shown in formula (I), a carbon atom marked with * is a chiral carbon atom with a configuration of R, S, or R/S, and the pyrimidine-fused diazepinone derivatives are levorotatory forms, dextrorotatory forms, or racemic forms;

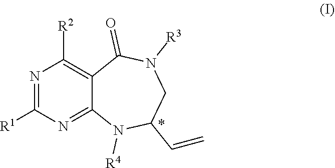

R¹ and R² are independently selected from hydrogen, halogen atom, hydroxyl group, carboxyl group, cyan group, nitro group, C1-C20 linear or branched alkyl group, C1-C20 fluoroalkyl group, C2-C20 alkenyl group, C2-C20 alkynyl group, C1-C20 alkoxy group, C3-C20 cycloalkyl group, C1-C20 amide group, C2-C20 ketone carbonyl group, C1-C20 sulfonyl group, C1-C9 alkyl silyl group, phenyl silyl group, amino group, C1-C20 N-alkyl substituted amino group, C1-C20 N,N-dialkyl substituted amino group, substituted or unsubstituted C3-C20 heterocyclic group or heterocyclic aryl group containing one or more of N, O, and S, substituted or unsubstituted aryl group, substituted or unsubstituted aryl methyl group;

The above-mentioned substituted substituents are independently selected from one or more combinations of hydrogen, halogen, hydroxyl, cyano group, nitro group, C1-C20 alkyl group, C1-C20 fluoroalkyl group, C1-C20 alkoxy group, amino group, C1-C20 N-alkyl substituted amino group or C1-C20 N,N-dialkyl substituted amino group;

One or more hydrogen atoms of the C1-C20 linear or branched alkyl group, the C1-C20 fluoroalkyl group, the C2-C20 alkenyl group, the C2-C20 alkynyl group, the C1-C20 alkoxy group, the C3-C20 cycloalkyl group, the C1-C20 amide group, the C2-C20 ketone carbonyl group, the C1-C20 sulfonyl group, the C1-C9 alkyl sill group, the phenyl silyl group, the amino group, the C1-C20 N-alkyl substituted amino group, the C1-C20 N,N-dialkyl substituted amino group, are substituted by fluorine atoms, chlorine atoms, bromine atoms, oxygen atoms, alkenyl groups, alkynyl groups, aryl groups, hydroxyl groups, amino groups, carbonyl groups, carboxyl groups, ester groups, cyano groups, methyl groups, ethyl groups, methoxy groups, intro groups.

Preferably, the above-mentioned aryl groups are independently C6-C20 aryl group respectively.

R³ and R⁴ are independently selected from hydrogen, C1-C20 linear or branched alkyl, C3-C20 cycloalkyl, C3-C20 cycloalkyl methylene group, C3-C20 allyl, C3-C20 propargyl group, C1-C20 acyl group, C1-C20 sulfonyl group, substituted or unsubstituted C3-C20 heterocyclic group or heterocyclic aryl group containing one or more of N, O and S, substituted or unsubstituted heterocyclic methyl group or heterocyclic aryl methyl group, substituted or unsubstituted C1-C20 alkoxycarbonyl group, substituted or unsubstituted aryl acyl group, substituted aryl sulfonyl group, substituted or unsubstituted aryl group, substituted or unsubstituted aryl methyl group;

The above-mentioned substituted substituents are independently selected from one or more combinations of hydrogen, C1-C20 alkyl group, C1-C20 fluoroalkyl group, halogen, nitro, C1-C20 alkoxy group, hydroxyl group, cyano group, C1-C20 N-alkyl substituted amino group or C1-C20 N,N-dialkyl substituted amino group;

One or more hydrogen atoms of the C1-C20 linear or branched alkyl group, the C3-C20 cycloalkyl group, the C3-C20 cycloalkyl methylene group, the C3-C20 allyl group, the C3-C20 propargyl group, the C1-C20 acyl group, the C1-C20 sulfonyl group, are substituted by fluorine atoms, chlorine atoms, bromine atoms, oxygen atoms, alkenyl groups, alkynyl groups, aryl groups, hydroxyl groups, amino groups ; carbonyl groups, carboxyl groups, ester groups, cyano groups, methyl groups, ethyl groups, methoxy groups, nitro groups.

Preferably, each of the above-mentioned aryl groups is independently C6-C20 acyl group.

Further, the structural formula of the pyrimidine-fused diazepinone derivatives is the formula (I) with a skeleton structure of the pyrimidine-fused diazepinone, R¹ and R² are independently selected from hydrogen, fluorine, chlorine, bromine, iodine, hydroxyl, carboxyl, cyano, nitro, methyl, ethyl, n-propyl, isopropyl, n-butyl, isobutyl, tent-butyl, trifluoromethyl, benzyl, methoxy, ethoxy, n-propoxy, isopropoxy, n-butoxy, isobutoxy, tert-butoxy, benzyloxy group, amino group, C1-C20 amide group, trimethylsilyl group, triethylsilyl group, triphenylsilyl group, C3-C20 heterocyclic group or heterocyclic aryl group containing one or more of N, O, and S, C1-C20 N-alkyl substituted amino group, C1-C20 N,N-dialkyl substituted amino group, C2-C20 ketone carbonyl group, C1-C20 sulfonyl group, substituted or unsubstituted aryl group; a substituent in the substituted aryl group is one or more combinations of C1-C20 alkyl, halogen, or C1-C20 alkoxy group; R³ and R⁴ are independently selected from hydrogen, C1-C20 linear or branched alkyl, C3-C20 cycloalkyl, C3-C20 cycloalkyl methylene group, C3-C20 heterocyclic group or heterocyclic aryl group containing one or more of N, O, and S, heterocyclic methylene group or heterocyclic aryl methylene group, allyl, propargyl, acetyl, benzoyl, C1-C20 sulfonyl group, tert-butoxycarbonyl group, fluorenyl methoxycarbonyl group, 2,2,2-trichloroethoxycarbonyl group, substituted or unsubstituted aryl group, substituted or unsubstituted aryl methylene group, substituted or unsubstituted benzenesulfonyl group; the substituted substituents are independently selected from one or more combinations of hydrogen, C1-C20 alkyl, C1-C20 fluoroalkyl, halogen, nitro or C1-C20 alkoxy group.

Further, the structural formula of the pyrimidine-fused diazepinone derivatives is the formula (I) with a skeleton structure of the pyrimidine-fused diazepinone: R¹ and R² are independently selected from hydrogen, fluorine, chlorine, bromine, iodine, methyl, ethyl, n-propyl, isopropyl, n-butyl, isobutyl, tert-butyl, trifluoromethyl group, methoxy, ethoxy, n-propoxy, isopropoxy, n-butoxy, isobutoxy, tert-butoxy, hydroxyl, carboxyl, cyano, cyclopentyl, cyclohexyl, amino, methylamino, ethylamino, diethylamine, diisopropylamino, trimethylsilyl, triethylsilyl, triphenylsilyl, acetamido, acetyl, trifluoroacetyl group, phenyl, anilino group, benzoyl, 3,4,5-trimethoxyphenyl, benzyl, 4-dimethylaminobenzyl, benzyloxy, methanesulfonyl, phenylsulfonyl, naphthyl, morpholinyl, pyrrolyl, tetrahydropyrrolyl, piperazinyl, 1-methylpiperazinyl, pyridyl, 4-methylpyridyl, methoxypyridyl, furyl, piperidinyl, 4-hydroxymethyl-3,5-dimethylpiperidinyl, thienyl, oxazolyl.

Further, the structural formula of the pyrimidine-fused diazepinone derivatives is the formula (I) with a skeleton structure of the pyrimidine-fused diazepinone: R³ and R⁴ are independently selected from hydrogen, methyl, ethyl, n-propyl, isopropyl, cyclohexyl, cyclopentylmethyl, allyl, propargyl, carbonyl, ethoxycarbonyl, tert-butoxycarbonyl, trichloroethoxyformyl, benzoyl, 4-bromobenzoyl, 9-fluorenylidenemethoxyformyl, 3-fluoro-4-(allylamido)phenyl, sulfonyl, tosyl, phenyl, 4-methoxyphenyl, 4-(trifluoromethypphenyl, 3,5-bis(trifluoromethyl)phenyl, benzyl, p-fluorobenzyl, 4-dimethylaminobenzyl, 4-methoxybenzyl, 2-tetrahydropyranyl, 4-(trifluoromethyl)benzyl, 3,5-bis(trifluoromethyl)benzyl, pyrimidyl, 4-fluoropyrimidinylmethyl, 4-chloropyrimidinylmethyl, pyrrotopyrimidinyl, morpholinyl, pyridyl, 3-methylpyridyl, pyridylmethyl, pyrazinyl, 4-trifluoro methyl pyrazinyl, piperazinyl, 3-methylpiperazinyl, piperidinyl, piperidylmethyl.

The present disclosure further provides a preparation method of the above-mentioned chiral or racemic pyrimidine-fused diazepinone derivatives, the method includes using pyrimidine allyl compound intermediates as raw materials, using iridium complexes formed by an interaction between iridium compounds and phosphoramidite ligands as catalysts, and obtaining the chiral or racemic pyrimidine-fused diazepinone derivatives by reaction under action of alkalis.

A reaction formula of the preparation method present disclosure is as shown in reaction formula (1):

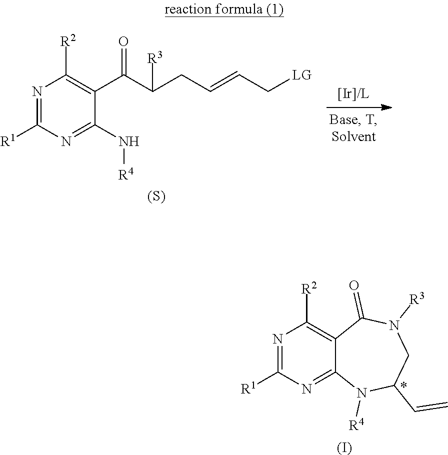

L is chiral or achiral ligands, Base are various alkalis and combinations of alkalis and additives, T is a reaction temperature, and Solvents are various organic solvents.

In the method of the present disclosure, the structural formula of the pyrimidine allyl compound intermediates is as shown in formula (S), LG is a leaving group, which is hydroxyl, chlorine, bromine,

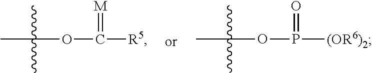

M is NH or O; R⁵ is a halogen-substituted or unsubstituted alkyl group, a halogen-substituted or unsubstituted C1-C20 alkoxy group; R⁶ is a C1-C20 alkyl group; or a substituted or unsubstituted aryl group.

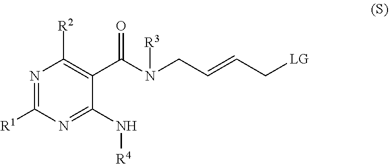

Preferably, in R⁵ of the formula (S), the halogen in the halogen-substituted C1-C20 alkyl group is fluorine, chlorine, bromine, or iodine. Further, the halogen-substituted C1-C20 alkyl group is trichloromethyl.

Preferably, in R⁶ of the formula (S), the substituent of the substituted aryl group is one or combinations of C1-C20 alkyl group, halogen, or C1-C20 alkoxy group. The aryl group is a C6-C20 aryl group.

Preferably, in R⁵ and R⁶ of the formula (S), the C1-C20 alkyl group is independently selected from methyl, ethyl, n-propyl, isopropyl, n-butyl, isobutyl, or tert-butyl. The C1-C20 alkoxy group is independently selected from methoxy, ethoxy, n-propoxy, isopropoxy, n-butoxy, isobutoxy, tert-butoxy, or benzyloxy.

The number of the substituents may be one or more, when there are multiple substituents, the substituents may be the same or different.

Further, the pyrimidine allyl compound intermediates are prepared by a method including the following steps: generating 2-chloro-4-substituted amino-6-substituent-pyrimidine-5-carboxylic acid methyl ester compounds by a reaction between 2,4-dichloro-6-substituent-pyrimidine-5-carboxylic acid methyl ester compounds and airline compounds, introducing a group R¹ through a conventional nucleophilic substitution reaction or coupling reaction, generating carboxyl compounds by hydrolysis, and obtaining the compounds of the formula S by reacting with

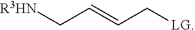

Compounds S are used as substrates for a further catalytic reaction to obtain product I. The reaction formula is as follows:

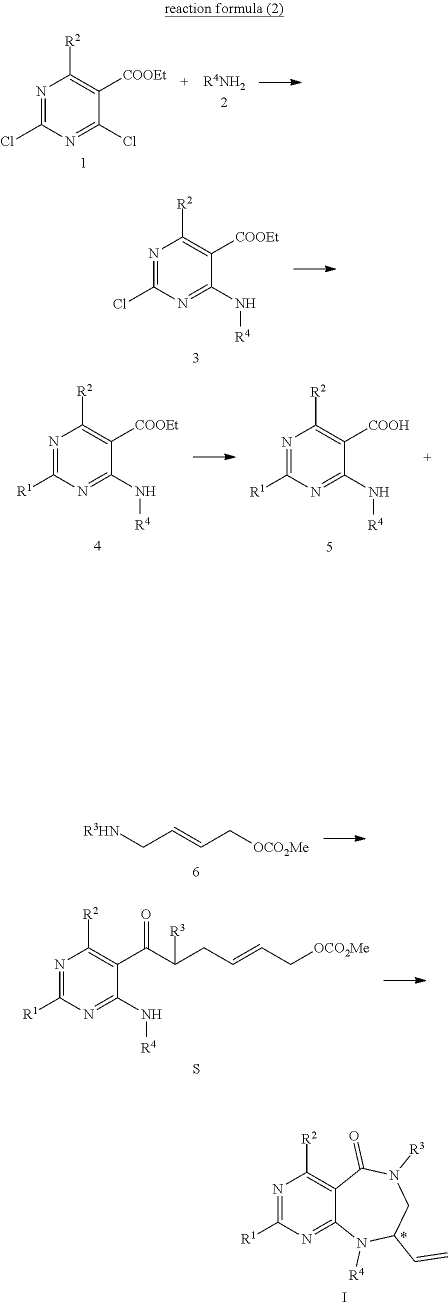

The 2,4-dichloro-6-substituent-pyrimidine-5-carboxylic acid methyl ester compounds may be 2,4-dichloro-pyrimidine-5-carboxylic acid methyl ester, 2,4-dichloro-6-hydroxy-pyrimidine-5-carboxylic acid methyl ester, 2,4-dichloro-6-cyano-pyrimidine-5-carboxylic acid methyl ester, 2,4-dichloro-6-phenyl-pyrimidine-5-carboxylic acid methyl ester, 2,4-dichloro-6-benzoyl-pyrimidine-5-carboxylic acid methyl ester, 2,4-dichloro-6-benzyl-pyrimidine-5-carboxylic acid methyl ester, 2,4-dichloro-6-amino-pyrimidine-5-carboxylic acid methyl ester, 2,4-dichloro-6-ethyl-pyrimidine-5-carboxylic acid methyl ester, 2,4-dichloro-6-methanesulfonyl-pyrimidine-5-carboxylic acid methyl ester, 2,4-dichloro-6-benzyloxy-pyrimidine-5-carboxylic acid methyl ester, 2,4-dichloro-6-triphenylsilyl-pyrimidine-5-carboxylic acid methyl ester, 2,4-dichloro-6-diethylamino-pyrimidine-5-carboxylic acid methyl ester, 2,4-dichloro-6-carboxy-pyrimidine-5-carboxylic acid methyl ester, 2,4-dichloro-6-N-methylpiperazine-pyrimidine-5-carboxylic acid methyl ester, etc.; the group R² can be directly introduced from commercial products or introduced by 2,4, 6-trichloro-irimidine-5-carboxylic acid methyl ester through the conventional nucleophilic substitution reaction, the Suzuki coupling reaction, or the Grignard reaction, to obtain the compounds 1.

In the method of the present disclosure, the alkalis can be organic alkalis or inorganic alkalis, such as triethylamine, 4-dimethylaminopyridine, 1,8-diazabicyclo[5,4,0] undecane-7-ene, 1.5-diazabicyclo[4,3,0]nona-5-ene, triethylenediamine, N,O-bis(trimethylsilyl)acetamide, cesium carbonate, potassium carbonate, lithium carbonate, potassium fluoride, sodium hydride, cesium fluoride, potassium phosphate, potassium acetate, sodium phosphate, sodium acetate, lithium acetate, n-butyllithium, sodium bis(trimethylsilyl)amide, lithium bis(trimethylsilyl)amide, potassium bis(trimethylsilyl)amide, sodium methoxide, sodium ethoxide, sodium isopropylate, proton sponge, lithium tert-butoxide, potassium tert-butoxide, sodium tert-butoxide, or diisopropylethylamine.

Further, the alkalis are added to the reaction system in combination with silver trifluorosulfonate, lithium chloride, or molecular sieve additives.

In the method of the present disclosure, a molar ratio of the pyrimidine allyl compound intermediates, the iridium atoms of the iridium compounds, the phosphoramidite ligands, and the alkalis is 1: (0.005-0.1): (0.005-0.2): (0.05-3).

In the method of the present disclosure, the reaction can be carried out at 0 to 120° C. A reaction time can be 20 min to 24 h.

In the method of the present disclosure, the iridium compounds can be at least one of [Ir(COD)Cl]₂, [Ir(dncot)Cl]₂, [Ir(OMe)(COD)]₂, [Ir(COD)₂]BArF₄, Ir(COD)₂BF₄, [Ir(OH)(COD)]₂, Ir(ppy)₃, [Ir(COD)₂]SbF₆ and the like.

In the present disclosure, the phosphoramidite ligands refers to the phosphoramidite ligands in a Chinese patent application with a publication number of CN109336887A, the details can be seen in paragraph [0076] of the description.

In the method of the present disclosure, the reaction product can be purified through a short silica gel column to obtain the chiral pyrimidine-fused diazepinone derivatives and their enantiomers or racemates.

In the method of the present disclosure, the reaction is carried out in an organic solvent system. The organic solvent may be a polar solvent or a non-polar solvent. Preferably, the organic solvent may be one or combinations of an aromatic hydrocarbon solvent or a substituted aromatic hydrocarbon solvent, a halogenated hydrocarbon solvent, an ether solvent, an amide solvent, an alkane solvent, a cycloalkane solvent, a nitrile solvent, dimethyl sulfoxide and alcoholic solvent. Further, the aromatic hydrocarbon solvent or substituted aromatic hydrocarbon solvent is preferably at least one of toluene, xylene, ethyl-benzene, cumene, chlorobenzene, and nitrobenzene; the halogenated hydrocarbon solvent is preferably at least one of dichloromethane, 1,2-dichloroethane, and chloroform; the ether solvent is preferably at least one of tetrahydrofuran, diethyl ether, ethylene glycol dimethyl ether, methyl tert-butyl ether, 1,4-dioxane; the amide solvent is preferably at least one of N,N-dimethylformamide, N,N-dimethylacetamide, N,N-dimethylproponamide, α-pyrrolidone, and N-methylpyrrolidone; the alkane solvent is preferably at least one of n-hexane, n-pentane and n-heptane; the cycloalkane solvent is preferably at least one of cyclopentane, cyclohexane, and cycloheptane; the nitrile solvent is preferably acetonitrile; the alcoholic solvent is preferably at least one of methanol, ethanol, isopropanol, n-propanol, and tert-butanol.

The preparation method of the present disclosure may include the following detailed steps:
(1) obtaining compounds 3 through a nucleophilic substitution reaction by using 2,4-dichloro-5-pyrimidinecarboxylic acid ethyl ester compounds 1 and amine compounds 2 as starting materials, and using N,N-diisopropylethylamine as alkali;
(2) obtaining a variety of substituted compounds 4 by a conventional coupling reaction or the nucleophilic substitution reaction between the compounds 3 and boric acid compounds, amine compounds, or sodium alkoxide compounds, in the presence of anhydrous solvents and alkalis;
(3) obtaining compounds 5 by a hydrolysis reaction of compounds 4 in water and in the presence of the alkalis;
(4) obtaining pyrimidine allyl compound intermediates by the compounds 5 being condensed with compounds 6 under action of 1-hydroxybenzotriazole (HOBt) and 1-ethyl-(3-dimethylaminopropyl) carbodiimide hydrochloride (EDCI), which is used as substrates for a subsequent catalytic allyl amination reaction;
(5) obtaining the compounds shown in the formula (I) by an intramolecular allyl amination reaction produced by metallic iridium complexes formed by iridium compounds and phosphoramidite ligands catalyzing the substrates in organic solvents in the presence of organic or inorganic alkalis.

In step (1), a molar ratio of the compounds I and the compounds 2 can be 1:1 to 1:1.2; a reaction temperature can be 50° C. to 100° C., and a reaction time can be 1 to 6 h.

In step (2), a molar ratio of the compounds 3, the boric acid compounds, amine compounds, sodium alkoxide compounds, and alkalis can be 1:(1-2):(1-2):(1-3); the reaction temperature can be 20° C. to 130° C., and the reaction time can be 30 min to 4 h.

In step (3), a molar ratio of the compounds 4 and alkalis can be 1:1 to 1:3; the reaction temperature can be 20° C. to 100° C., and the reaction time can be 10 mm to 2 h; the alkalis can be sodium hydroxide, potassium hydroxide, or lithium hydroxide, etc.

In step (4), a molar ratio of the compounds 5, the compounds 6, HOBt, and EDCI can be 1:1: (1-2): (1-2); the reaction temperature can be 0 to 80° C.; the reaction time can be 1 to 10 h; the reaction is carried out in a solvent, such as dichloromethane, tetrahydrofuran, dioxane, N,N-dimethylformamide, N,N-dimethylacetamide, or dimethyl sulfoxide.

In step (5), a molar ratio of substrate S, iridium, ligand, and alkali can be 1: (0.005-0.1): (0.005-0.2): (0.05-3); the reaction temperature can be 0 to 70° C.; the reaction time can be 3 min to 24 h.

The preparation method of the present disclosure uses iridium-phosphoramidite ligand complexes as catalysts, especially when iridium-chiral bridged phosphoramidite ligand complexes are used as the catalysts, through the careful design and synthesis of the pyrimidine allyl carbonate substrates, a method for synthesizing the pyrimidine-fused diazepinone derivatives with high efficiency and high enantioselectivity through the intramolecular allyl amination reaction is provided; the enantiomers are obtained by preparing corresponding catalysts from ligands with opposite configurations, and carrying out a similar catalytic intramolecular allylic amination reaction; the racemates are obtained by preparing the corresponding catalyst from the racemic ligands, and carrying out a similar catalytic intramolecular allelic amination reaction.

The present disclosure further provides the application of the above-mentioned chiral or racemic pyrimidine-fused diazepinone derivatives or pharmaceutically acceptable salts thereof in the preparation of drugs or their lead compounds for preventing or treating depression.

Compared with the existing technology, the present disclosure has the following advantages and beneficial effects:

The present disclosure provides a strategy and a method for the synthesis of novel chiral pyrimidine-fused diazepinone derivatives with high efficiency, high regioselectivity, and high enantioselectivity, which effectively uses iridium-phosphoramidite ligand complexes as catalysts to prepare a variety of chiral pyrimidine-fused diazepinone derivatives by means of carefully designed and synthesized pyrimidine allyl substrates and catalytic intramolecular allyl amination reactions.

At the same time, the present disclosure further carries out preliminary in vitro and in vivo antidepressant activity evaluations on the prepared chiral or racemic pyrimidine-fused diazepinone derivatives. The results indicate that: the derivatives of the present disclosure have good inhibitory effects on GR, cofilin-1, and NF-κB protein, and can reverse the apoptosis of PC12 cells caused by corticosterone in a depressed state, which indicates that the pyrimidine-fused diazepinone derivatives of the present disclosure have good antidepressant activities.

Compared with existing methods, the preparation method of the present disclosure can be applied to many different types of pyrimidine allyl compounds, and has high catalytic activity, mild reaction conditions, wide substrate application range, simple operation, and good reaction yield (up to 99%), high enantioselectivity (up to 99% ee). This method of efficiently constructing chiral pyrimidine-fused diazepinone derivatives by catalyzing the asymmetric intramolecular allylic amination reaction and the corresponding derivatives synthesized therefrom have not yet been reported in the literature at home and abroad.

The present disclosure not only enriches the application of chiral-bridged phosphoramidite ligands and other types of phosphoramidite ligands, but also broadens the applicable scope of allylation reaction substrates, and provides novel chiral heterocyclic molecules and a new efficient construction method for the development of new drugs.

### BRIEF DESCRIPTION OF THE DRAWINGS

FIG. 1 is a structural view of a single crystal of I-9 prepared in Embodiment 3.

FIG. 2 is a schematic view showing a relationship between a concentration and time of antidepressant effects of compounds of the present disclosure. The concentration used in B is 0.625 uM, and the concentration used in C and D is 1.0 uM.

FIG. 3 is a schematic view showing inhibitory effects of the compounds of the present disclosure on overexpression of GR, cofilin-1, and NF-κB proteins. The concentration used in A and B is 0.625 uM, and the concentration used in C and D is 1.0 uM.

FIG. 4 is a schematic view showing antidepressant effects of compounds with different chiral configurations. The concentration used in A is 1.0 uM, and the concentration used in B, C, and D is 0.625 uM.

FIG. 5 is a schematic view showing experimental results of the derivatives of the present disclosure on reserpine-induced depression model mice. A is a mouse tail suspension test; B is a mouse forced swimming test; C is a mouse sucrose preference test; D is a mouse open field test; E is a single-dose mouse tail suspension test; F is a single-dose forced swimming experiment in mice.

### DETAILED DESCRIPTION

The present disclosure will be described in further detail below with reference to examples, but the implementation of the present disclosure is not limited thereto. The materials involved in the following embodiments can be obtained from commercial sources unless otherwise specified. The methods described are conventional methods unless otherwise stated. The dosage of each component is in molar volume parts, mol, L.

### Embodiment 1: Preparation of Pyrimidine Allyl Compounds (S)

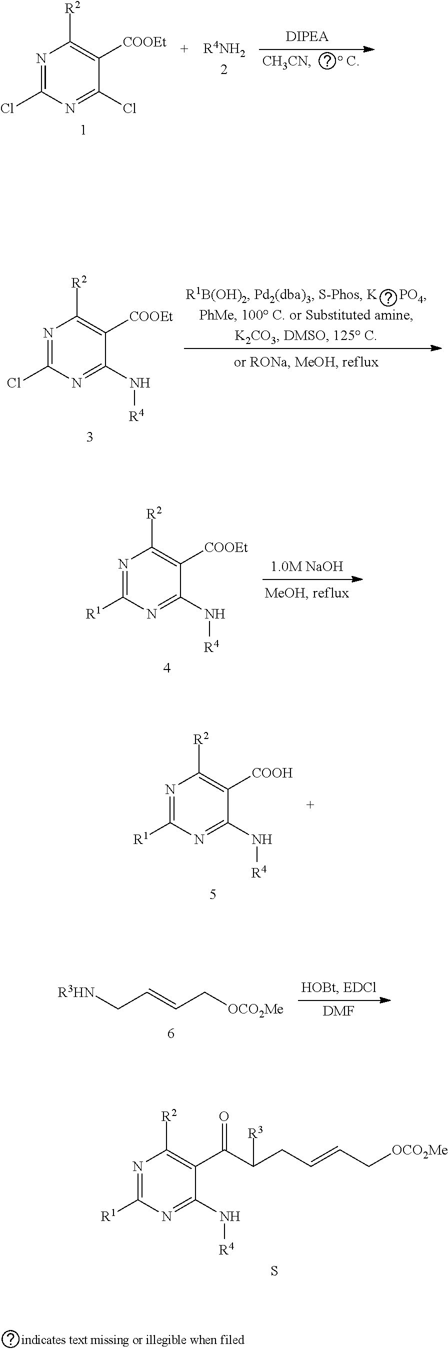

Compounds 3 are obtained by a stir reaction performed at 80° C. by using 2,4-dichloro-5-pyrimidinecarboxylic acid ethyl ester compounds 1 (1.0 equivalent) and amine compounds 2 (1.05 equivalent) as starting materials, N,N-diisopropylethylamine (2.0 equivalent) as alkali, acetonitrile as solvent, and then through a nucleophilic substitution reaction; compounds 4 are obtained by a conventional Suzuki coupling reaction or the nucleophilic substitution reaction between the compounds 3 and boric acid compounds (2.0 equivalent), amine compounds (1.2 equivalent), or sodium alkoxide compounds (3.0 equivalent), in the presence of anhydrous solvents and alkalis; compounds 5 are obtained by a hydrolysis reaction of the compounds 4 in the presence of methanol solvent and sodium hydroxide (1.0 mol/L); the compounds 5 are dissolved with DMF, then 1-hydroxybenzotriazole (HOBt, 1.1 equivalent) and 1-ethyl-(3-dimethylaminopropyl) carbodiimide hydrochloride (EDCI, 1.1 equivalent) are added into the dissolved compound 5 respectively, the reaction is stirred at room temperature for 30 minutes, then compounds 6 (1.1 equivalent) are added into the reaction solution, and the reaction is continued at room temperature with stirring, after the reaction of raw materials is complete, the reaction solution is poured into ice water, and then is extracted with ethyl acetate, the combined organic phase is concentrated under reduced pressure, and the crude product is purified by column chromatography, to obtain the pyrimidine allyl intermediates S.

Products of the pyrimidine allyl intermediates 5:

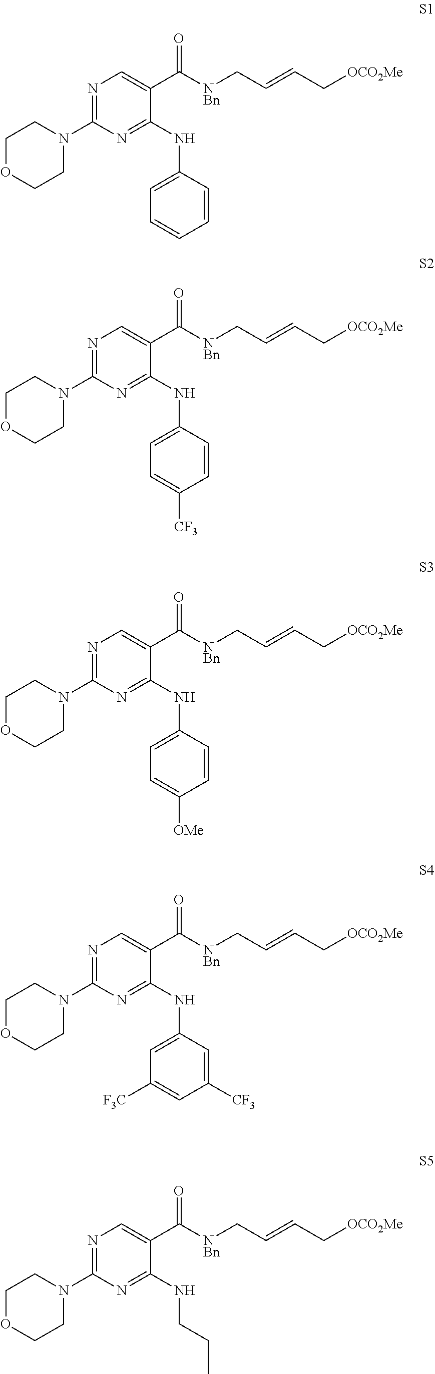

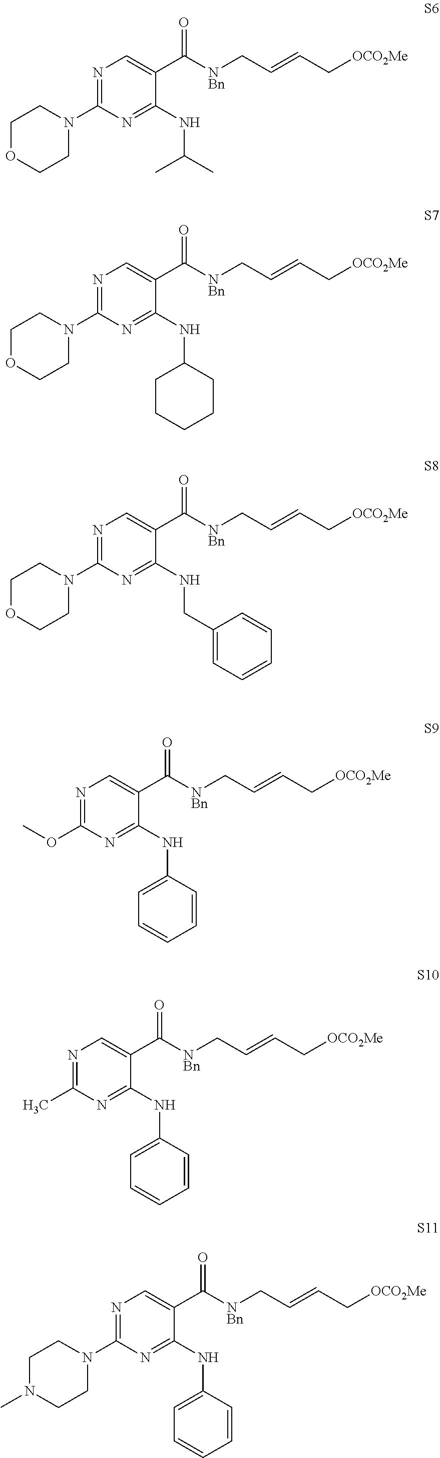

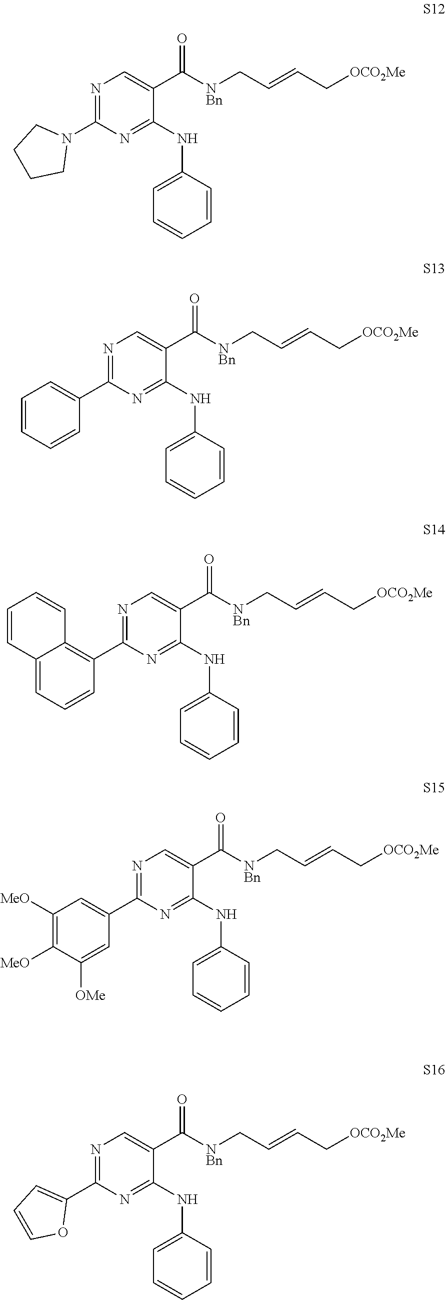

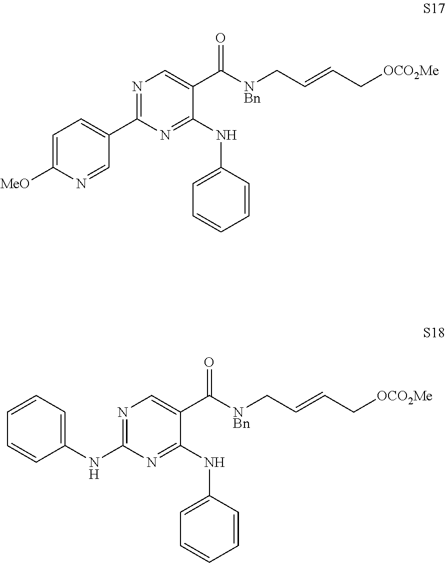

### S1: (E)-4-(N-benzyl-2-morpholinyl-4-anilinopyrimidine-5-carboxamido)-2-butene carbonate methyl ester

White oil, 0.93 g, yield 90%. ¹H NMR (400 MHz, CDCl₃) δ 9.55 (s, 1H), 8.15 (s, 1H), 7.67-7.54 (m, 2H), 7.40-7.28 (m, 5H), 7.28-7.23 (m, 1H), 7.12-7.04 (m, 1H), 5.92-5.82 (m, 1H), 5.78-5.68 (m, 1H), 4.71 (s, 2H), 4.65 (dd, J=5.8, 1.2 Hz, 2H), 4.04 (d, J=5.6 Hz, 2H), 3.86-3.77 (m, 7H), 3.76-3.70(m, 4H). ¹³C NMR (100 MHz, CDCl₃) δ 170.51, 160.79, 159.61, 156.55, 155,53, 138.76, 136.42, 129.76, 128,95, 128.95, 128.69, 128.69, 128.69, 127.69, 127.40, 127,37, 123.31, 121.23, 121.23, 100.02, 67.34, 66.80, 66.80, 54.91, 50.58, 48.65, 44.30, 44.30. HRMS (ESI) calcd for C₂₈H₃₁N₅O₅ [M+H]⁺: 518.2398, found: 518.2393.

### S2: (E)-4-(N-benzyl-2-morpholinyl-4-(4-trifluoromethylanilino)pyrimidine-5-carboxamido)-2butene carbonate methyl ester

Light yellow oil, 1.08 g, yield 93%. ¹H NMR (400 MHz, CDCl₃) δ 9.89 (s, 1H), 8.19 (s, 1H), 7.72 (d, J=8.1 Hz, 2H), 7.59 (d, J=8.5 Hz, 2H), 7.47.34 (m, 2H), 7.33-7.23 (m, 3H), 5.93-5.82 (m, 1H), 5.80-5.64 (m, 1H), 4.71 (s, 2H), 4.65 (dd, J=5.8, 1.2 Hz, 2H), 4.05 (d, J=5.5 Hz, 2H), 3.85-3.80 (m, 4H), 3.79 (s, 3H), 3.78-3.71 (m, 4H). ¹³C NMR (100 MHz, CDCl³) δ 169.25, 159.63, 158.46, 155.81, 154.47, 140.99, 135,26, 128.49, 127.91, 127.91, 126.67, 126.49, 126.31, 126.31., 124.87, 124.87, 123.48 (q, J=32.8 Hz), 121.99, 119.36, 119.36, 99.18, 66.21, 65.64, 65.64, 53.78, 49.55, 47.68, 43.29, 43.29, ¹⁹F NMR (376 MHz, CDCl³) δ−61.74. HRMS (ESI) calcd for C₂₉H₃₀F₃N₅O₅ [M+H]⁺: 586.2272, found: 586.2265.

### S3: (E)-4-(N-benzyl-4-(4-methoxyanilino)-2-morpholinopyrimidine-5-carboxamido)-2-butene carbonate methyl ester

White oil, 0.93 g, yield 85%. ¹H NMR (400 MHz, CDCl₃) δ 9.37 (s, 1H), 8.12 (s, 1H), 7.50-7.44 (m, 2H), 7.39-7.33 (m, 2H), 7.33-7.28 (m, 1H), 7.28-7.23 (m, 2H), 7.05-6.82 (m, 2H), 5.94-5.82 (m, 1H), 5.78-5.68 (m, 1H), 4.70 (s, 2H), 4.68-4.63 (m, 2H), 4.04 (d, J=5.6 Hz, 2H), 3.82 (s, 3H), 3.79 (s, 3H), 3.79-3,74 (m, 4H), 3.74-3.68 (m, 4H), ¹³C NMR (101 MHz, CDCl₃) δ 170.58, 160.79, 159.70, 156.41, 155.86, 155.52, 136.47, 131.79, 129.82, 129.82, 128.9 128.92, 127.65, 127.39, 127.31, 123,09, 123.09, 113.85, 113.85, 99.71, 67.34, 66.79, 66.79, 55.48, 54.89, 50.63, 48.65, 44.23, 44.23. HRMS (ESI) calcd for C₂₉H₃₃N₅O₆ [M+H]⁺: 548.2504, found: 548.2508.

### S4: (E)-4-(N-benzyl-4-(3,5-ditrifluoromethylanilino)-2-morpholinopyrimidine-5-carboxamido)-2-butene methyl carbonate ester

Light yellow oil, 1.04 g, yield 80%. ¹H NMR (400 MHz, CDCl₃) δ 10.14 (s, 1H), 8.25 (s, 1H), 8.18 (s, 2H), 7.55 (s, 1H), 7.43-7.36 (m, 2H), 7.35-7.24 (m, 3H), 5.98-5.85 (m, 1H), 5.84-5.69 (m, 1H), 4.74 (s, 2H), 4.68 (dd, J=5.8, 1,3 Hz, 2), 4.08 (d, J=5.6 Hz, 2H), 3.87-3.81 (m, 4H), 3.80 (s, 3H), 3.79-3.71 (m, 4H), ¹³C NMR (101 MHz, CDCl₃) δ 169.95, 160.36, 159.37, 157.15, 155.45, 140.36, 136.25, 131.62 (q, J=33.1 Hz), 129.31, 128.85, 127.65, 127.29, 124.75, 122.04, 120.61 (q, J=4.2 Hz), 119.33, 115.75, 115.71, 115.68, 115.64, 115.60, 100.09, 67.10, 67.10, 66.48, 54.62, 50.10, 48.70, 44.31, 44.31. ¹⁹F NMR (376 MHz, CDCl₃) δ−63.09. HRMS (ESI) calcd for C₃₀H₂₉F₆N₅O₅ [M+H]⁺: 654.2146, found: 654.2130.

### S5: (E)-4-(N-benzyl-2-morpholinyl-4-propylaminopyrimidine-5-carboxamido)-2-butene carbonate methyl ester

Light yellow oil, 0.79 g, yield 82%. ¹H NMR (400 MHz, CDCl₃) δ 8.00 (s, 1H), 7.39-7.32 (m, 2H), 7.32-7.20 (m, 4H), 5.90-5.79 (m, 1H), 5.76-5.64 (m, 1H), 4.74-4.60 (m, 4H), 4.01 (d, J=5.3 Hz, 2H), 3.86-3.78 (m, 7H), 3.76-3.70 (m, 4H), 3.46-3.34 (m, 2H), 1.71-1.59 (m, 2H), 0.99 (t, J=7.4 Hz, 3H). ¹³C. NMR (101 MHz, CDCl₃) δ 170.51, 161.59, 160.85, 155.50, 155.40, 136.73, 129.98, 12.8.71, 128.71, 127.41, 127.31, 127.31, 126.97, 99.56, 67.24, 66.69, 66.69, 54.68, 50.81, 48.41,44.06, 44.06, 42.15, 22.44, 11.60. HRMS (ESI) calcd for C₂₅H₃₃N₅O₅ [M+H]⁺: 484.2554, found: 484.2546.

### S6: (E)-4-(N-Benzyl-4-isopropylamine-2-morpholinopyrimidine-5-carboxamido)-2-butene carbonate methyl ester

Light yellow oil, 0.82 g, yield 85%. ¹H NMR (400 MHz, CDCl₃) δ 8.02 (s, 1H), 7.99 (s, 1H), 7.40-7.30 (m, 2H), 7.26-7.16 (m, 2H), 7.03 (d, J=7.2 Hz, 1H), 5.83 (dt, J=16.0, 5.6 Hz, 1H), 5.69 (dt, J=15.9, 6.1 Hz, 1H), 4.69-4.55 (m, 4H), 4.24 (h, J=6.6 Hz, 1H), 3.99 (d, J=5.7 Hz, 2H), 3.83-3.68 (m, 1H), 1.24 (d, J=6.5 Hz, 6H). ¹³C NMR (101 MHz, CDCl₃) δ 170.59, 160.90, 160.71, 155.61, 155.47, 136.68, 130.06, 128.79, 128.79, 127.50, 127.34, 127.34, 127.02, 99.58, 67.32, 66.78, 66.78, 54.81, 54.76, 50.44, 48.46, 44.11, 44.11 42.03, 22.53. HRMS (EST) calcd for C₂₅H₃₃N₅O₅ [M+H]⁺: 484.2554, found: 484.2559.

### S7: (E)-4-(N-benzyl-4-cyclohexylamino-2-morpholinopyrimidine-5-carboxamido)-2-butene carbonate methyl ester

Light yellow oil, 0.90 g, yield 86%. ¹H NMR (400 MHz, CDCl₃) δ 7.98 (s, 1H), 7.38-7.27 (m, 3H), 7.24-7.19 (m, 2H) 7.11 (d, J=7.5 Hz, 1H), 5.88-5.78 (m, 1H), 5.74-5.62 (m, 1H), 4.71-4.56 (m, 4H), 3.98 (d, J=5.6 Hz, 2H), 3.96-3.88 (m, 1H), 3.80 (s, 3H), 3.79-3.75 (m, 4H), 3.75-3.69 (m, 4H), 2.09-1.89 (m 2H), 1.80-1.68 (m, 2H), 1.66-1.55 (m, 1H), 1.47-1.16 (m, 5H ). ¹³C NMR (101 MHz, CDCl₃) δ 170.42, 160.83, 160.58, 155,60, 155.31, 136.80, 129.95, 128.60, 128,60, 127.30, 127.27, 127.27, 126.93, 99.48, 67.13, 66.58, 66.58, 54.51, 50.27, 48.66, 48.42, 44,00, 44,00, 32.41, 32.41, 25.69, 5.49, 24.59. HRMS (ESI) calcd for C₂₈H₃₇N₅O₅[M+H]⁺: 524.2867, found: 524.2853.

### S8: (E)-4-(N-benzyl-4-benzyl-2-morpholinopyrimidine-5-carboxamido)-2-butene carbonate methyl ester

Light yellow oil, 0.93 g, yield 88%. ¹H NNW (400 MHz, CDCl₃) δ 8.02 (s, 1H), 7.63 (t, J=5.7 Hz, 1H), 7.38-7.28 (m, 5H), 7.26-7.17 (m, 5H), 5.90-5.78 (m, 1H), 5.74-5.62 (m, 1H), 4.69-4.54 m, 4H), 3.99 (d, J=5.6 Hz, 3H), 3.80 (s, 2H), 3.78-3.72 (m, 4H), 3.69 (q, J=4.7 Hz. 4H). ¹³ C NMR (101 MHz, CDCl₃) δ 170.29, 161.52, 160.82, 155.77, 155.42, 139.45, 136.90, 129.98, 128.73, 128.43, 128.43, 127.42, 127.39, 127.35, 127.35, 127.33, 127.33, 127.05, 126.94, 99.84, 67.23, 66.57, 66.57, 54.62, 50.33, 48.47, 44.24, 44.11, 44.11, HRMS (ESI) calcd for C₂₉H₃₃N₅O₅ [M+H]⁺: 532.2554, found: 532.2544.

### S9: (E)-4-(N-benzyl-2-methoxy-4-anilinopyrimidine-5-carboxamido)-2-butene carbonate methyl ester

Light yellow oil, 0.87 g, yield 94%. ¹H NMR (400 MHz, CDCl₃) δ 9.42 (s, 1H), 8.24 (s, 1H), 7.70-7.60 (m, 2H), 7.40-7.32. (m, 5H), 7.30-7.22 (m, 2H), 7.12 (ddt, J=8.5, 7.0, 1.2 Hz, 1H), 5.92-5.81 (m, 1H), 5.80-5.69 (m, 1H), 4.71 (s, 2H), 4.68-4.60 (m, 2H), 4.06 (d, J=5.5 Hz, 2H), 3.97 (s, 34), 3.79 (s, 3H). ¹³C NMR (101 MHz, CDCl₃) δ 169.49, 165.46, 160.80, 157.09, 155.51, 138.09, 135.99, 129.20, 129.05, 129.05, 128.87, 128.87, 128.41, 127.89, 127.74, 123.99, 121.33, 121.33, 104.39, 67.20, 63.88, 54.99, 54.94, 50.46, 48.85, HRMS (EST) calcd for C₂₅H₂₆N₄O₅ [M+H]⁺: 463.1976, found: 463.1964.

### S10: (E)-4-(N-benzyl-2-methyl-4-anilinopyrimidine-5-carboxamido)-2-butene carbonate methyl ester

Light yellow oil, 0.84 g, yield 95%. ¹H NMR (400 MHz, CDCl₃) δ 8.99 (s, 1H), 8.29 (s, 1H), 7.68 (d, J=8.0 Hz, 2H), 7.42-7.30 (m, 4H), 7.28-7.20 (m, 2H), 7.11 (t, J=7.4 Hz, 1H), 5.85 (dt, J=15.9, 5.5 Hz, 1H), 5.74 (dt, J=15.6, 5.7 Hz, 1H), 4.71 (s, 2H), 4.64 (d, J=5.4 Hz, 2H), 4.05 (d, J=5.4 Hz, 2H), 3.79 (s, 3H), 2.57 (s, 3H). ¹³C. NMR (101 MHz, CDCl₃) δ 168.71, 168.56, 162.24, 158.12, 155.26, 153.97, 138.52, 136.12, 128.94, 128.76, 128.76, 128.61, 128.61, 127.62, 127.62, 123.41, 120.92, 120.92, 108.01, 66.94, 54.57, 35,97, 31.00, 26.02. HRMS (EST) calcd for C₂₅H₂₆N₅O₄ [M+H]⁺: 447,2027, found: 447.2031.

### S11: (E)-4-(N-benzyl-2-(4-methyl-1-piperazinyl)-4-anilinopyrimidine-5-carboxamido)-2-butene carbonate methyl ester

White oil, 1.02 g, yield 96%. ¹H NMR (400 MHz, CDCl₃) δ 9.56 (s, 1.14), 8.14 (s, 1H), 7.63-7.56 (m, 2H), 7.40-7.30 (m, 5H), 7.30-7.23 (m, 2H), 7.11-7.04 (m, 1H), 5.93-5.82 (m, 1H), 5.79-5.65 (m, 1H), 4.70 (s, 2H), 4.65 (dd, J=5.8, 1.3 Hz, 2H), 4.04 (d, J=5.6 Hz, 2H), 3.87 (t, J=5.1 Hz, 4H), 3.79 (s, 3H), 2.48 (t, J=5.0 Hz, 4H), 2.35 (s, 3H), ¹³C NMR (101 MHz, CDCl₃) δ 170.59, 160.64, 159.58, 156.63, 155.50, 138.89, 136.48, 129.81, 128.91, 128.91, 128.65, 128.65, 127.64, 127.40, 127.40, 127.32, 127.32, 123.15, 121.12, 121.12, 99.68, 67.31, 54.89, 54.86, 50.57, 48.67, 46.15, 43.77, 43.77, HRMS (EST) calcd for C₂₉H₃₄N₆O₄ [M+H]⁺: 531.2714, found: 531.2710.

### S12: (E)-4-(N-benzyl-4-anilino-2-tetrahydropyrroylpyrimidine-5-carboxamido)-2-butene carbonate methyl ester

White oil, 0.95 g, yield 95%. ¹H NMR (400 MHz, CDCl₃) δ 9.57 (s, 1H), 8.08 (s, 1H), 7.76-7.62 (m, 2H), 7.32-7.12 (m, 7H), 6.99-6.91 (m, 1H), 5.88-5.73(m, 1H), 5.71-5.55 (m, 1H), 4.62 (s, 2H), 4.56 (dd, J=5.9, 1.3 Hz, 2H), 3.95 (d, J=5.6 Hz, 2H), 3.69 (s, 3H), 3.60-3.46 (m, 4H), 1.93-1.83 (m, 4H), ¹³C NMR (101 MHz, CDCl₃) δ 170.93, 159.32, 159.23, 156.45, 155.52, 139.30, 136.50, 129.88, 128.91, 128.91, 128.62, 128.62, 127.62, 127.42, 127.42, 127.27, 122.78, 120.65, 120.65, 99.05, 67.38, 54.88, 50.58, 48.68, 47.07, 46.52, 25.58, 25.30. HRMS (EST) calcd for C₂₈H₃₁N₅O₄ [M+H]⁺: 502.2449, found: 502.2447.

### S13: (E)-4-(N-benzyl-2-phenyl-4-anilinopyrimidine-5-carboxamido)-2-butene carbonate methyl ester

Light yellow oil, 0.93 g, yield 92% ¹H NMR (400 MHz, CDCl₃) δ 9.13 (s, 1H), 8.48 (s, 1H), 8.42-8,34 (m, 2H), 7.79-7.71 (m, 2H), 7.47 (dd, J=5.8, 1.7 Hz, 3H), 7.45-7.39 (m, 31-1), 7.39-7.34 (m, 2H), 7.34-7.26 (m, 3H), 7.18-7.12 (m, 1H), 5,88 (dt, J=15.7, 5.5 Hz, 1H), 5.77 (dt, J=15.6, 5.7 Hz, 1H), 4.76 (s, 2H), 4.66 (dd, J=5.7, 1.2 Hz, 2H), 4.16-4.02 (m, 2H), 3.79 (s, 3H). ¹³C NMR (101 MHz, CDCl₃) δ 169.06, 164.65, 158.74, 155.52, 154.66, 138.56, 137.44, 136.08, 131.19, 129.12, 129.05, 129.05, 128.92, 128.65, 128.56, 127.91, 127.83, 123.82, 121.32, 121.32, 108.45, 77.80, 77.48, 77.16, 67.20, 67.20, 60.38, 54.90, 54.90, 53.69. HRMS (ESI) calcd for C₃₀H₂₈NO₄[M+H]⁺: 509.2183, found: 509.2171.

### S14: (E)-4N-benzyl-2-(1-naphthyl)-4-anilinopyrimidine-5-carboxamido)-2-butene carbonate methyl ester

Light yellow oil, 0.98 g. yield 88%. ¹H NMR (400 MHz, CDCl₃) δ 9.18 (s, 1H), 8.86-8.79 (m, 1H), 8.58 (s, 1H), 8.13 (dd, J=7.2, 1.3 Hz, 1H), 7.96 (d, J=8.1 Hz, 1H), 7.92-7.87 (m, 1H), 7.77-7.71 (m, 2H), 7.59-7.53 (m, 1H), 7.52-7.46 (m, 2H), 7.42-7.36 (m, 2H), 7.36-7.28 (m, 5H), 7.13-7.06 (m, 1H), 5.98-5.85 (m, 1H), 5.85-5.76 (m, 1H), 4.80 (s, 2H), 4.68 (dd, J=5.6, 1.3 Hz, 2H), 4.15 (d, J=5.3 Hz, 2H), 3.80 (s, 3H). ¹³C NMR (101 MHz, CDCl₃) δ 169.14, 167.21, 158.66, 155.54, 154.22, 138.44, 135.57, 134.12, 131.05, 130.92, 129.59, 129.12, 129.12, 129.04, 128.94, 128.94, 128.70, 128.44, 128.00, 127.96, 127.53, 126.67, 126.36, 125.87, 125.12, 124.19, 123.85, 121.23, 121.23, 107.94, 67.19, 62.54, 54.97. HRMS (ESI) calcd for C₃₄H₃₀N₄O₄ [M+H]⁺: 559.2340, found: 559.7.337.

### S15: (E)-4-(N-benzyl-4-anilino-2-(3,4,5-trimethoxyphenyl)pyrimidine-5-carboxamido)-2-butene carbonate methyl ester

Light brown oil, 1.08 g, yield 90%. ¹H NMR (400 MHz, CDCl₃) δ 9.10 (s, 1H), 8.46 (s, 1H), 8.01 (s, 2H), 7.79-7.66 (m, 3H), 7.38 (m, 3H), 7.35-7.28 (m, 2H), 7.17-7.10 (m, 1H), 5.89 (dt, J=15.9, 5.5 Hz, 1H), 5.77 (dt, J=15.6, 5.7 Hz, 1H), 4.76 (s, 2H), 4.69-4.62 (m, 2H), 4.14-4.08 (m, 2H), 3.94 (s, 6H), 3.91 (s, 3H), 3.80 (s, 3H). ¹³C NMR (101 MHz, CDCl₃) δ 168.91, 163.78, 163.76, 162.48, 158.44, 155.41, 154.53, 153.03, 153.03, 140.69, 138.37, 135.94, 132.50, 129.03, 128.95, 128.95, 128.50, 128.50, 127.82, 127.77, 123.85, 121.58, 121.58. 108.13, 105.53, 105.53, 67.10, 60.79, 55.91, 55.91, 54.81, 36.35, 31.29. HRMS (EST) calcd for C₃₃H₃₄N₄O₇ [M+]⁺: 599.2500, found: 599.2485.

### S16: (E)-4-(N-benzyl-2-(2-furyl)-4-anilinopyrimidine-5-carboxamido)-2-butene carbonate methyl ester

Light brown oil, 0.94 g, yield 95%. ¹H NMR (400 MHz, CDCl₃) δ 9.21 (s, 1H), 8.43 (s, 1H), 8.01 (s, 1H), 7.75 (m, 2H), 7.61 (d, J==1.8 Hz, 1H), 7.44-7.31 (m, 4H), 7.30 -7.20 (m, 3H), 7.14 (t, J=7.4 Hz, 1H), 6.55 (dd, J=3.5, 1.7 Hz, 1H), 5.97-5.82 (m, 1H), 5.81-5.66 (m, 1H), 4.73 (s, 2H), 4.65 (d, J=5.5 Hz, 2H), 4.09 (d, J=5.4 Hz, 2H), 3.79 (s, 3H), ¹³C NMR (101 MHz, CDCl₃) δ 162.50, 158.47, 157.29, 155.46, 154.58, 151.98, 145.45, 138.36, 135.85, 129.03, 129.03, 128.94, 128.83, 128.83, 127.89, 127.84 127.33, 123.80, 121.01, 121.01, 114.59, 112.27, 112.27, 107.87, 67.11, 54.88, 50.48, 48.60. HRMS (ESI) called for C₂₈H₂₆N₄O₅ [M+H]⁺: 499.1976, found: 499.1965.

### S17: (E)-4-(N-benzyl-2-(4-methoxy-3-pyridyl)-4-anilinopyrimidine-5-carboxamido)-2-butene carbonate ethyl ester

Transparent oil, 1.02 g yield 95%. ¹H NMR (400 MHz, CDCl₃) δ 9.18 (d, J=2.3 Hz, 1H), 9.14 (s, 1H), 8.50 (dd, J=8.7, 2.4 Hz, 1H), 8.43 (s, 1H), 7.74-7.66 (m, 2H), 7.46-7.35 (m, 4H), 7.35-7.27 (m, 3H), 7.19-710 (m, 1H), 6.80 (d, J=8.8 Hz, 1H), 5.93-5.84 (m, 1H), 5.77 (dt, J=15.6, 5.7 Hz, 1H), 4.75 (s, 2H), 4.71-4.62 (m, 2H), 4.10 (dd, J=5=6.5, 2.9 Hz, 2H), 4.01 (s, 3H), 3.80 (s 3H). ¹³C, NMR (101 MHz, CDCl₃) δ 169.08, 165.93, 1.63,20, 158.66, 155.51, 154.55, 148.59, 138.65, 138.26, 135.91, 134.55, 129.08, 129.08, 128.94, 128.94, 127.96, 127.88, 127.88, 127.47, 126.69, 123.94, 121.34, 121.34, 110.61, 108.10, 67.17, 62.46, 60.41, 54.95, 53.85. HRMS (ESI) calcd for C₃₀H₂₉N₅O₅ [M+H]⁺: 540.2241, found: 540.2233.

### S18: (E)-4-(N-Benzyl-2,4-dipanilinopyrimidine-5-carboxamido)-2-butene carbonate methyl ester

Light yellow oil, 0.94 g, yield 90%. ¹H NMR (400 MHz, CDCl₃) δ 9.50 (s, 1H), 8.18 (s, 1H), 7.67-7.57 (m, 2H), 7.56-7.50 (m, 2H), 7.40-7.20 (m, 9H), 7.18-7.10 (m, 1H), 7.08-7.00 (m, 1H), 5.95-5.82 (m, 1H), 5.82-5.64 (m, 1H), 4.72-2H), 4.64 (dd, J=5.9, 1.2 Hz, 2H), 4.06 (d, J=5.6 Hz, 2H), 3.78 (s, 3H). ¹³C NMR (101 MHz, CDCl₃) δ 170.10, 160.05, 159.69, 156.27, 155.59, 139.26, 138.50, 136.14, 129.61, 129.01, 129.01, 128.78, 128.78, 128.76, 128.76, 127.80, 127.59, 127.52, 123.98, 123.15, 122.27, 122.27, 120.81, 120.81, 120.81, 101.89, 67.33, 54.92, 50.62, 48.86. HRMS (ESI) calcd. for C₃₀H₂₉N₅O₄ [M+H]⁺: 524.2292, found: 524.2281.

### Embodiment 2

Phosphoramidite ligands L1-L9 can be prepared according to conventional methods in this field, the details can refer to the literature: (a) J. Am. Chem. Soc. 2011, 133, 4785. (b) J. Am. Chem. Soc 2012, 134, 4812. (c) J. Am, Chem. Soc 2012, 134, 15022. (d) J. Org. Chem. 2015, 80, 6968. (e) J. Am. Chem. Soc. 2015, 137, 553. (f) Organometallics 2016, 35, 2467. (g) ACS Catal, 2017, 7, 2397. (h) J. Am. Chem. Soc. 2017, 139, 15022. (i) Org. Lett. 2019, 21, 608.

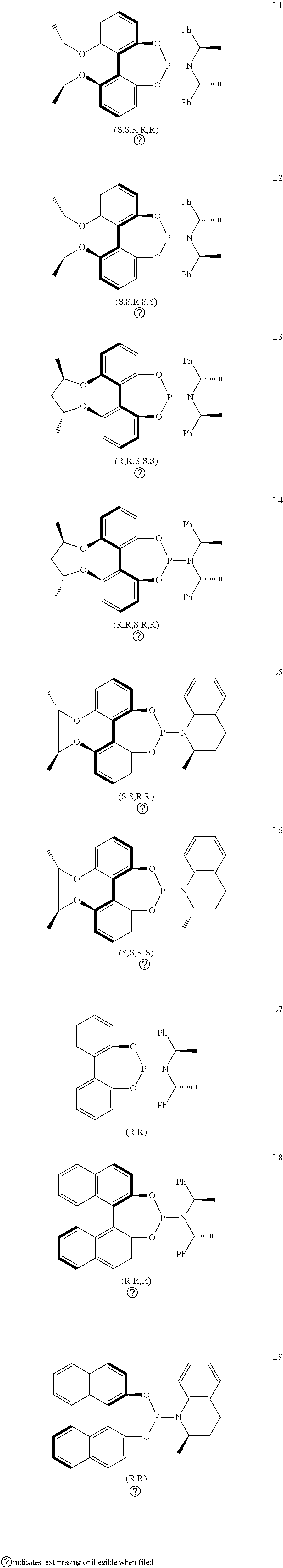

### Embodiment 3

The chiral pyrimidine-fused diazepinone derivatives are prepared by an intramolecular allyl amination reaction that uses the pyrimidine allyl intermediates S as substrates and is catalyzed by iridium-phosphoramidite complexes, the preparation method includes:

under nitrogen protection, adding iridium compounds (0.004 mole parts), phosphoramidite ligands L1-L9, n-propylamine (0.5 volume parts), and tetrahydrofuran (1 volume part) in sequence, stirring at 50° C. for 30 minutes, and then cooling to room temperature, removing the solvent under reduced pressure, then adding the substrates S (0.2 mole parts), the alkalis (0.22 mole part), and organic solvents (2.0 volume part) in sequence, performing the stirring reaction at 25-80° C., and purifying the reaction product by column chromatography to obtain the allyl amination products I.

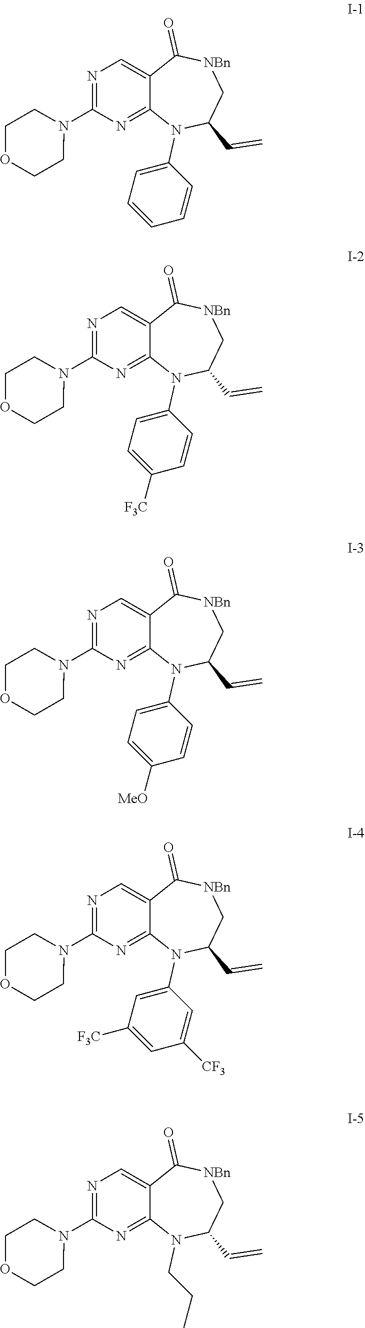

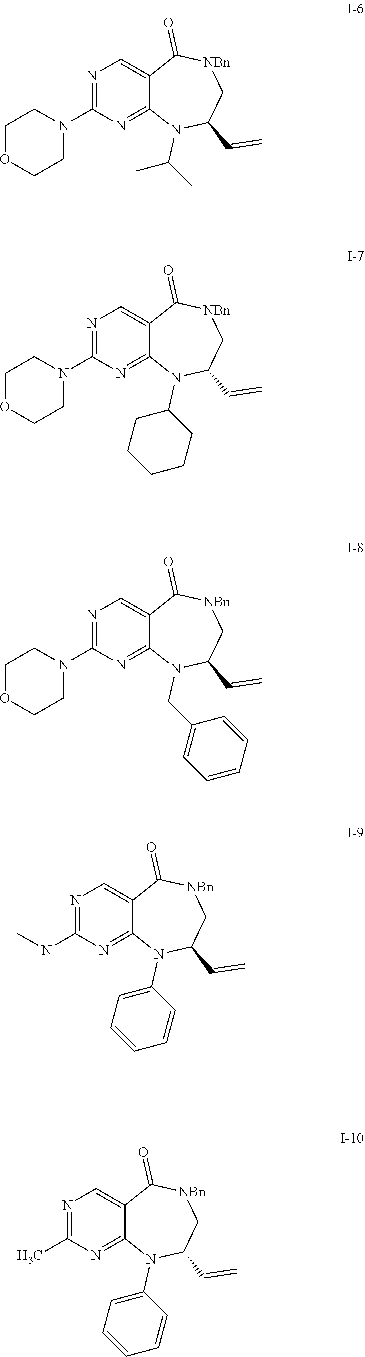

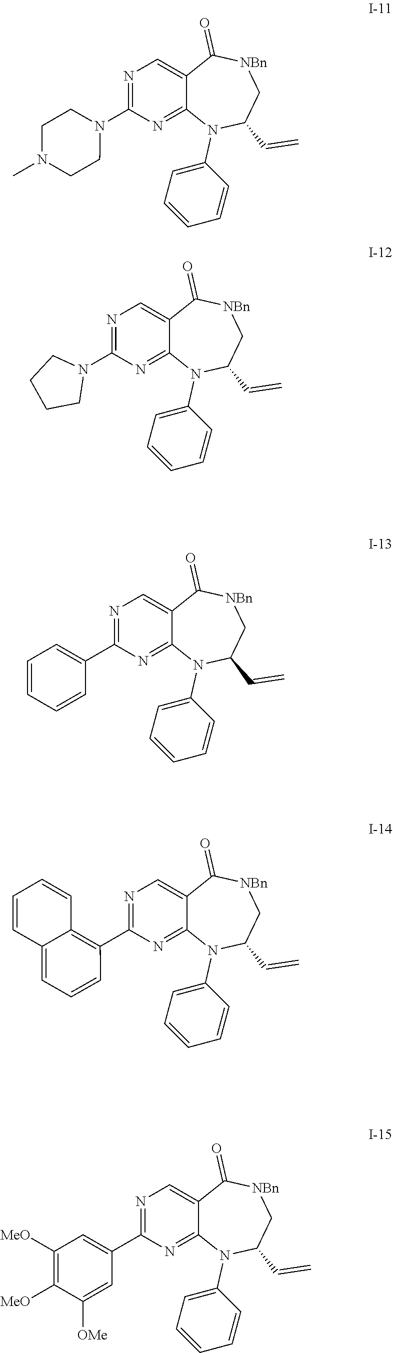

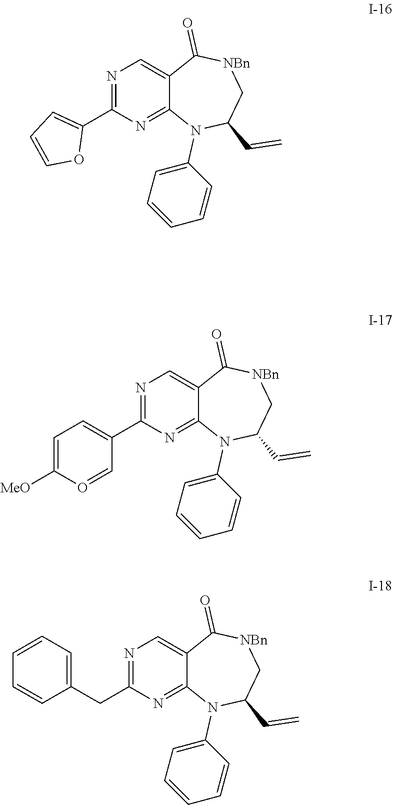

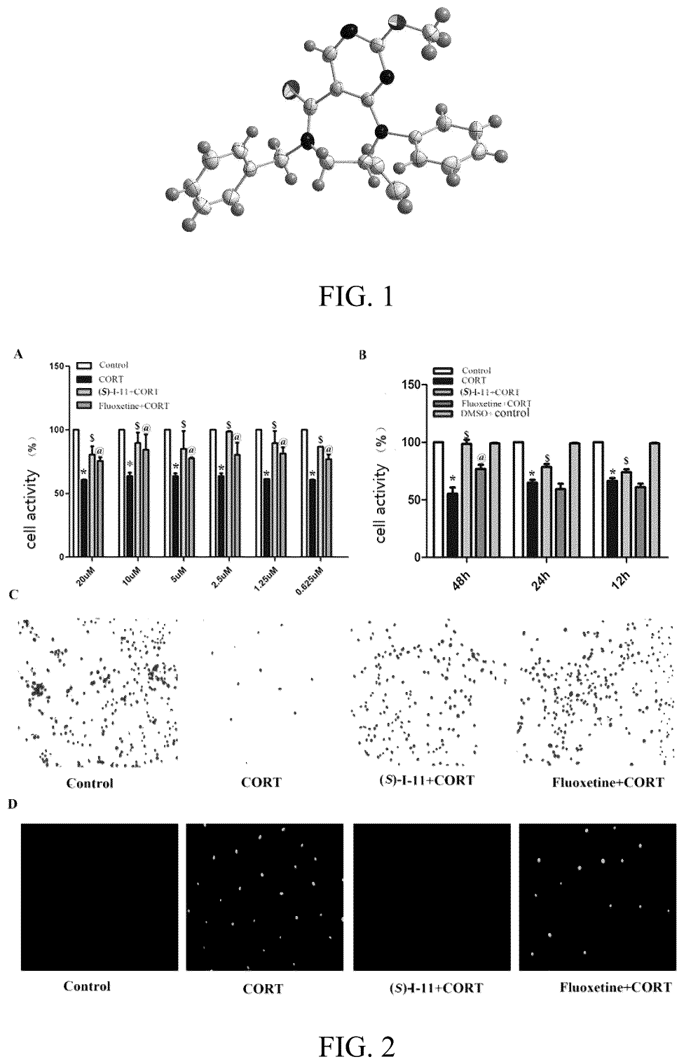

Compounds I-1 to I-18 are prepared as above, and specific reaction conditions can be referred to Table 1. FIG. 1 is a structure view of a single crystal of compound I-9 prepared in Embodiment 3. The molar ratio in Table 1 refers to a molar ratio of substrate S: iridium: ligand: alkali.

TABLE 1
Reaction conditions of compound I-1 to compound I-18

| | | Iridium | | | | Molar | Temperature | Time |
|---|---|---|---|---|---|---|---|---|
| Compound | Substrate S | compounds | Ligand | Alkali | Solvent | ratio | (° C.) | (h) |
| I-1 | R¹ is morpholinyl; | [Ir(COD)Cl]₂ | L5 | DBU | DME | 1:0.02:0.04:1 | 25 | 4 |
| | R² is H; R³ is Bn; | | | | | | | |
| | R⁴ is Ph; LG is OCO₂Me | | | | | | | |
| I-2 | R¹ is morpholinyl; | [Ir(COD)Cl]₂ | L1 | DABCO | THF | 1:0.02:0.04:2 | 20 | 8 |
| | R² is H; R³ is Bn; | | | | | | | |
| | R⁴ is p-trifluoromethylphenyl; | | | | | | | |
| | LG is OTroc | | | | | | | |
| I-3 | R¹ is morpholinyl; | [Ir(COD)₂]SbF₆ | L3 | BSA | MTBE | 1:0.02:0.04:2 | 30 | 6 |
| | R² is H; R³ is Bn; | | | | | | | |
| | R⁴ is p-methoxyphenyl; | | | | | | | |
| | LG is OCO₂Me | | | | | | | |
| I-4 | R¹ is morpholinyl; | [Ir(COD)Cl]₂ | L6 | K₂CO₃ | dioxane | 1:0.02:0.04:2 | 50 | 10 |
| | R² is H; R³ is Bn; | | | | | | | |
| | R⁴ is 3,5- | | | | | | | |
| | ditrifluoromethylphenyl; | | | | | | | |
| | LG is OCO₂Me | | | | | | | |
| I-5 | R¹ is morpholinyl; | [Ir(COD)₂]SbF₆ | L2 | K₃PO₄ | CH₃CN | 1:0.02:0.04:2 | 50 | 2 |
| | R² is H; R³ is Bn; | | | | | | | |
| | R⁴ is n-propyl; LG is OBz | | | | | | | |
| I-6 | R₁ is morpholinyl; | [Ir(COD)Cl]₂ | L4 | DIPEA | DME | 1:0.02:0.04:2 | 25 | 20 |
| | R² is H; R³ is Bn; | | | | | | | |
| | R⁴ is isopropyl; LG is OPiv | | | | | | | |
| I-7 | R¹ is morpholinyl; | [Ir(OMe)(COD)]₂ | L7 | KF | DME | 1:0.03:0.06:2 | 50 | 12 |
| | R² is H; R³ is Bn; | | | | | | | |
| | R⁴ is cyclohexyl; LG is OAc | | | | | | | |
| I-8 | R¹ is morpholinyl; | [Ir(OMe)(COD)]₂ | L5 | BSA | DCE | 1:0.02:0.04:2 | 30 | 14 |
| | R² is H; R³ is Bn; | | | | | | | |
| | R⁴ is Bn; LG is OBoc | | | | | | | |
| I-9 | R¹ is methoxy; | [Ir(COD)Cl]₂ | L5 | DBU | DME | 1:0.02:0.04:3 | 30 | 4 |
| | R² is H; R³ is Bn; | | | | | | | |
| | R⁴ is Ph; LG is OCO₂Me | | | | | | | |
| I-10 | R¹ is methyl; | [Ir(COD)Cl]₂ | L2 | Na₂CO₃ | O-xylene | 1:0.02:0.04:2 | 40 | 12 |
| | R² is H; R³ is Bn; | | | | | | | |
| | R⁴ is Ph; LG is OCO₂Me | | | | | | | |
| I-11 | R¹ is N-methylpiperazinyl; | [Ir(COD)₂]BArF₄ | L3 | CsF | CH₃CN | 1:0.03:0.06:2 | 50 | 10 |
| | R² is H; R³ is Bn; | | | | | | | |
| | R⁴ is Ph; LG is OAc | | | | | | | |
| I-12 | R¹ is tetrahydropyrrolyl; | [Ir(OMe)(COD)]₂ | L9 | K₃PO₄ | THF | 1:0.02:0.04:2 | 30 | 16 |
| | R² is H; R³ is Bn; | | | | | | | |
| | R⁴ is Ph; LG is Troc | | | | | | | |
| I-13 | R¹ is phenyl; | [Ir(OH)(COD)]₂ | L4 | KOAC | MTBE | 1:0.02:0.04:1 | 20 | 18 |
| | R² is H; R³ is Bn; | | | | | | | |
| | R⁴ is Ph; LG is OCO₂Me | | | | | | | |
| I-14 | R¹ is naphthyl; | Ir(COD)₂BF₄ | L2 | KF | O-xylene | 1:0.01:0.02:1 | 50 | 12 |
| | R² is H; R³ is Bn; | | | | | | | |
| | R⁴ is Ph; LG is OCO₂Me | | | | | | | |
| I-15 | R¹ is 3,4,5-trimethoxyphenyl; | [Ir(COD)Cl]₂ | L8 | DBN | DME | 1:0.02:0.04:2 | 25 | 8 |
| | R² is H; R³ is Bn; | | | | | | | |
| | R⁴ is Ph; LG is OCO₂Me | | | | | | | |
| I-16 | R¹ is 2-furyl; | [Ir(COD)₂]BArF₄ | L6 | DBU | DME | 1:0.02:0.04:2 | 30 | 10 |
| | R² is H; R³ is Bn; | | | | | | | |
| | R⁴ is Ph; LG is OCO₂Me | | | | | | | |
| I-17 | R¹ is 4-methoxy-3-pyridyl; | [Ir(OMe)(COD)]₂ | L2 | K₃PO₄ | dioxane | 1:0.02:0.04:1 | 30 | 14 |
| | R² is H; R³ is Bn; | | | | | | | |
| | R⁴ is Ph; LG is OBoc | | | | | | | |
| I-18 | R¹ is aniline; | [Ir(COD)Cl]₂ | L5 | DIPEA | DCM | 1:0.02:0.04:2 | 20 | 00 |
| | R² is H; R³ is Bn; | | | | | | | |
| | R⁴ is Ph; LG is OCO₂Me | | | | | | | |

In Table 1, LG is a leaving group, Bn is a benzyl group, DIPEA is diisopropylethylamine, DMAP is 4-dimethylaminopyridine, DABCO is triethylenediamine, and DBU is 1,8-diazabicycloundec-7-ene, BSA is N,O-bistrimethylsilylacetamide, DBN is 1,5-diazabicyclo[4.3.0]non-5-ene, Ac represents acetyl, Boc represents tert-butoxycarbonyl, Piv represents 2,2-dimethylpropionyl, Bz represents benzoyl, Troc represents 2.2.2-trichloroethoxycarbonyl, DME represents ethylene glycol dimethyl ether, MTBE represents methyl tert-butyl ether.

Compound I-1: (R)-6-benzyl-2-morpholino-9-phenyl-8-vinyl-6,7,8,9-tetrahydro-5H-pyrimido [4,5-e][1,4]diazapine-5-one: white solid, 84 mg, yield 95%; MP: 125.4-125.5° C.; 98% ee; Ee value is detected by chiral chromatographic column (Daicel Chiralcel IA-3, mobile phase, n-hexane/2-propanol=80/20, flow rate: 1 ml/min, detection temperature: 30° C., detection wavelength: 254 nm, retention time: 11.084 min, 14.645 min), [α]_{D}²⁰=−27.4° (c=0.15, CHCl₃). ¹H NMR (400 MHz, CDCl₃) δ 8.95 (s, 1H), 7.39-7.27 (m, 7H), 7.26-7.20 (m, 1H), 7.14-7.06 (m, 2H), 5.76 (ddd, J=16.9, 10.4, 6.4 Hz, 1H), 5.44 (d, J=11.8 Hz, 1H), 5.29-5.08 (m, 2H), 4.56-4.45 (m, 1H), 4.10 (d, J=14.9 Hz, 1H), 3.93 (d, J=15.2 Hz, 1H), 3.69-3.45 (m, 9H). ¹³C NMR (101 MHz, CDCl₃) δ 167.68, 164.36, 160.75, 158.37, 145.52, 137.21, 132.95, 129.00, 129.00, 128.69, 128.69, 128.33, 128.33, 127.86, 127.86, 127.59, 126.53, 118.84, 102.64, 66.70, 66.70, 66.63, 52.70, 50.20, 43.95, 43.95. HRMS (EST) calcd for C₂₆H₂₇N₅O₂ [M+H]⁺: 442.2238, found: 442.2241.

Compound I-2: (S)-6-benzyl-2-morpholinyl-9-(4-trifluoromethylphenyl)-8-vinyl-6,7,8,9-tetrahydro-5H-pyrimido[4,5-e][1,4]diazepin-5-one: white solid, 91 mg, yield 90%; MP: 153.0-153.4° C.; 92% ee; Ee value is detected by chiral chromatographic column (other parameters are the same as compound I-1, retention time: 11.660 min, 20.165 min), [α]_{D}²⁰+27.3° (c=0.15, CHCl₃). ¹H NMR (400 MHz, CDCl₃) δ 8.97 (s, 1H), 7.62 (d, J=8.5 Hz, 2H), 7.40-7.26 (m, 5H), 7.2-7.21 (m, 2H), 5.77 (ddd, J=16.9, 10.4, 6.4 Hz, 1H), 5.41 (d, J=14.8 Hz, 1H), 5.29-5.15 (m, 2H), 4.51 (ddt, 6.4, 5.0, 1.4 Hz, 1H), 4.17 (d, J=14.9 Hz, 1H), 3.92 (d, J=15.2 Hz, 1H), 3.62 (dd, J=15.2, 6.1 Hz, 9H). ¹³C NMR (101 MHz, CDCl₃) δ 167.39, 164.33, 160.87, 158.36, 148.65, 137.03, 132.84, 132.84, 128.73, 128.73, 128.33, 128.33, 127.69, 127.54, 127.54, 126.02 (q, J=3.8 Hz), 125.34, 122.64, 119.18, 103.65, 66.63, 66.63, 66.34, 52.47, 50.11, 43.99, 43.99. ¹⁹F NMR (376 MHz, CDCl₃) δ −62.25. HRMS (ESI) calcd. for C₂₇H₂₆F₃N₅O_{2 [M+H]}⁺: 510.2111, found: 510.2103.

Compound I-3: (R)-6-benzyl-9-(4-methoxyphenyl)-2-morpholinyl-8-vinyl-6,7,8,9-tetrahydro-5H-pyrimido[4,5-e][1,4]diazepine-5-one: white solid, 67 mg, yield 72%; MP: 189.6-190.4° C.; 83% ee; Ee value is detected by chiral chrornatographic column (Daicel Chiraicel IF-3, mobile phase, n-hexane/2-propanol=75/25, flow rate: 1 mL/min, detection temperature: 30° C., detection wavelength: 254 nm, retention time: 31.325 min, 33.575 min), [α]_{D}²⁰=−18.5° (c=0.10, CHCl₃). ¹H NMR (400 MHz, CDCl₃) δ 8.96 (s, 1H), 7.41-7.24 (m, 5H), 7.08-6.98 (m, 2H), 6.93-6.79 (m, 2H), 5.78 (ddd, J=17.0, 10.3, 6.5 Hz, 1H), 5.46 (d, J=14.8 Hz, H), 5.34-5.09 (m, 2H), 4.45 (t, J=5.9 Hz, 1H), 4.11 (d, J=14.9 Hz, 1H), 3.94 (d, J=15.2 Hz, 1H), 3.83 (s, 3H), 3.68-3.41 (m, 9H). ¹H C NMR (101 MHz, CDCl₃) δ 167.73, 164.32, 160.83, 158.53, 157.82. 138.39, 137.24, 133.04, 132.05, 131.93, 128.90, 128.67, 128.67, 128.45, 128.32, 127.57, 118.76, 114.12, 102.56, 67.04, 66.72, 66.72, 55.44, 52.68, 50.16, 43.98, 43.98. HRMS (ESI) calcd for C₂₇H₂₉N₅O₃ [M+H]⁺: 472.2343, found: 472.2347.

Compound I-4 : (R)-6-benzyl-9-(3,5-ditrifluoromethylphenyl)-2-morpholinyl-8-vinyl-6,7,8,9-tetrahydro-5H-pyrimido[4,5-e][1,4]diazepin-5-one: white solid, 95 mg, yield 82%; MP: 153.3-153.4° C.; 89% ee; Ee The value is detected by chiral chromatographic column (other parameters are the same as compound I-1, retention time: 5.540 min, 10.430 min), [α]_{D}²⁰=−31.2° (c=0.20, CDCl₃). ¹H NMR (400 MHz, CDCl₃) δ 8.98 (s, 1H), 7.69 (s, 1H), 7.61 (d, J=1.6 Hz, 2H), 7.37-7.27 (m, 5H), 5.73 (ddd, J=16.8, 10.4, 6.2 Hz, 1H), 5.38-5.14 (m, 3H), 4.56-4.47 (m, 1H), 4.23 (d, J=14.8 Hz, 1.14), 3.88 (d, J=15.1 Hz, 1H), 3.71-3.44 (m, 9H). ¹³C NMR (101 MHz, CDCl₃) δ 167.04, 164.68, 160.68, 157.88, 146.54, 136.89, 132.64, 132.06 (q, J=33.4 Hz), 128.77, 128.77, 128.42, 128.42, 127.80, 127.80, 127.30, 124.39, 121.68, 119.54, 119.54, 119.14, 103.87, 66.57, 66.11, 66.11, 52.41, 50,08, 44.00, 44.00. ¹⁹F NMR (376 MHz, CDCl₃) δ−62.93. HRMS (ESI) calcd for C₂₈H₂₅F₆N₅O₂ [M+H]⁺: 578,1985, found: 578.1971.

Compound I-5: (S)-6-benzyl-2-morpholino-9-propyl-8-vinyl-6,7,8,9-tetrahydro-5H-pyrimido[4,5-e][1,4]diazepine-5-one: light yellow oil, 63 mg, yield 78%. 86% ee; Ee value is detected by chiral chromatographic column (other parameters are the same as compound I-1, retention time: 12.215 min, 14.187 min), [α]_{D}^{°}=+20.5° (c=0.10, CHCl₃). ¹H NMR (400 MHz, CDCl₃) δ0 8.85 (s, 1.4), 7.50-6.93 (m, 5H), 5.64 (ddd, J=17.1, 10.3, 5.8 Hz, 1H), 5.36 (d, J=14.9 Hz, 1H), 5.22, (d, J=10.3 Hz, 1H), 5.13 (d, J=17.0 Hz, 2H), 4.12 (t, J=5.4 Hz, 1H), 4.05 (d, J=14.9 Hz, 1H), 3,91-3.77 (m, 4H), 3.81-3.69 (m, 4H), 3.65 (d, J=15.3 Hz, 1H), 3.48 (dd, J=15.3, 5.9 Hz, 3.05 (ddd, J=13.6, 9.8, 5,6 Hz, 1H), 1.78-1.38 (m, 2H), 0.88 (t, J=7.4 Hz, 3H). ¹³C NMR (101 MHz, CDCl₃) δ 167.98, 163.74, 161.31, 157.48, 137.25, 132.78, 128.63, 128.63, 128.26, 128.26, 127.51, 118.18, 102.73, 66.86, 66.86, 64.08, 52.47, 51.63, 49.53, 44.30, 44.30, 20.23, 11.56. HRMS (ESI) calcd for C₂₃H₂₉N₅O₂ [M+H]⁺: 408.2402, found: 408.2401.

Compound I-6: (R)-6-benzyl-9-isopropyl-2-morpholinyl-8-vinyl-6,7,8,9-tetrahydro-5H-pyrimido[4,5-e][1,4]diazepine-5-one: light yellow oil, 46 mg, yield 56%. 80% ee; (mobile phase, n-hexane/2-propanol=85/15, other parameters are the same as compound I-1, retention time: 12.218 min, 19.029 min), [α]_{D}²⁰=−13.4° (c=0.12, CHCl₃). ¹H NMR (400 MHz, CDCl₃) δ 8.92 (s, 1H), 7.52-6.94 (m, 5H), 5.73-5.60 (m, 1H), 4.88-4.35 (m, 1H), 3.89-3.68 (m, 4H), 3.62-3.53 (m, 4H), 3.32-3.24 (m, 1H), 3.10-3.03 (m, 1H), 2.02-1.94 (m, 2H), 1.36 (d, J=6.7 Hz, 6H), ¹³C NMR (101 MHz, CDCl₃) δ 169.30, 160.04, 159.13, 156.68, 136.77, 128.75, 128.75, 128.60, 128.51, 127.48, 127.32, 127.32, 99.70, 67.08, 66.84, 66.84, 53.88, 53.83, 48.92, 44.08, 44.08, 22.50, 22.50. HRMS (ESI) calcd for C₂₃H₂₉N₅O₂ [M+H]⁺: 408.2394, found: 408.2387.

Compound I-7: (S)-6-benzyl-9-cyclohexyl-2-morpholinyl-8-vinyl-6,7,8,9-tetrahydro-5H-pyrimido[4,5-e][1,4]diazepin-5-one: light yellow oil, 74 mg, yield 83%. 84% ee; (Other parameters are the same as compound I-1, retention time: 13.320 min, 16.457 min), [α]_{D}²⁰=−12.6° (c=0.10, CHCl₃), ¹H NMR (400 MHz, CDCl₃) δ 7.99 (s, 1H), 7.43-6.87 (m, 5H), 5.87-5.56 (m, 1H), 4.93-4.47 (n, 4H), 4.02-3.86 (m, 3H), 3.81-3.59 (m, 8H), 3.21-3.06 (m, 1H), 2.07-1.96 (m, 2H), 1.83-1.48 (m, 3H), 1.46-1.14 (m, 5H). ¹³C NMR (101 MHz, CDCl₃) δ 170.64, 161.00, 160.68, 155.69, 136.80, 128.79, 128.79, 128.63, 128.55, 127.50, 127.45, 127.45, 99.74, 66.87, 66.87, 66.87, 64.25, 48.95, 44.14, 44.14, 42.80, 32.64, 32.64, 25.81, 24.81, 24.81. HRMS (ESI) calcd for C₂₆H₃₃N₅O_{2 [M+H]}⁺: 448.2716, found: 448.2714.

Compound I-8: (R)-6,9-dibenzyl-2-morpholinyl-8-vinyl-6,7,8,9-tetrahydro-5H-pyrimido[4,5-e][1.4]diazepine-5-one: light yellow oil, 90 mg, yield 88%. 90% ee; Ee value is detected by chiral chromatographic column (other parameters are the same as compound I-3, retention time: 15.127 min, 22.066 min), [α]_{D}²⁰=−21.2° (c=0.12, CHCl₃). ¹H NMR (400 MHz, CDCl₃) δ 8.89 (s, 1H), 7.39-7.14 (m, 10H), 5.77-5.62 (m, 2H), 5.40 (d, J=14.9 Hz, 1H), 5.29 (d, J=10.2 Hz, 1H), 5.18 (d, J=17.1 Hz, 1H), 4.09-4.03 (m, 1H), 4.02 (d, J=4.2 Hz, 1H), 3.98 (d, J=4.9 Hz, 1H), 3.80-3.69 (m, 4H), 3.68-3.58 (m , 5H), 3.41 (dd, J=15.4, 5.8 Hz, 1H). ¹³C NMR (101 MHz, CDCl₃) δ 167.79, 163.68, 161.08, 157.97, 137.76, 137.14, 132.39, 128.65, 128.65, 128.65, 128.64, 128.64, 128.30, 128.30, 127.56, 127.26, 127.26, 118.73, 102.66, 66.74, 66.74, 62.82, 52.43, 51.35, 49.34, 44.36, 44.36. HRMS (ESI) calcd for C₂₇H₂₉N₅O₂ [M+H]⁺: 456.2394, found: 456.2394.

Compound I-9: (R)-6-benzyl-2methoxy-9-phenyl-8-vinyl-6,7,8,9-tetrahydro-5H-pyrimido[4,5-e][1,4]diazepin-5-one: white solid, 71 mg, yield 92%; MP: 136.7-136.9° C. 96% ee; Ee value is detected by chiral chromatographic column (mobile phase, n-hexane/2-propanol=75/25, detection temperature: 25° C., other parameters are the same as compound I-1, retention time: 10.080 min, 12.555 min), [α]_{D}²⁰=−22.1° (c=0.12, CHCl₃). ¹H NMR (400 MHz, CDCl₃) δ 9.08 (s, 1H), 7.44-7.34 (m, 4H), 7.34-7.27 (m, 4H), 7.17-7.11 (m, 2H), 5.77 (ddd, J=16.9, 10.3, 6.4 Hz, 1H), 5.46 (d, J=14.8 Hz, 1H), 5.31-5.12 (m, 2H), 4.60-4.53 (m, 1H), 4.14 (d, J=14.8 Hz, 1H), 3.96 (d, J=15.4 Hz, 1H), 3.65 (dd, J=15.4, 6.0 Hz, 1H), 3.57 (s, 3H). ¹³C NMR (101 MHz, CDCl₃) δ 166.99, 166.05, 165.45, 159.25, 144.81, 136.81, 132.23, 129.31, 129.31, 128.77, 128.77, 128.40, 128.40, 127.77, 127.71, 127.71, 127.07, 119.26, 106.48, 67.06, 54.40, 52.88, 49.97. HRMS (ESI) calcd for C₂₃H₂₂N₄O₂ [M+H]⁺: 387.1816, found: 387.1805.

Compound I-10: (S)-6-benzyl-2-methyl-9-phenyl-8-vinyl-6,7,8,9-tetrahydro-5H-pyrimido[4,5- e][1,4]diazepin-5-one: white solid, 58 mg, yield 78%. MP: 152.8-153.0° C. 84% ee Ee value is detected by chiral chromatographic column (other parameters are the same as compound I-1, retention time: 11.343 min, 15.262 min), [α]_{D}²⁰=+18.4° (c=0.12, CHCl₃). ¹H NMR (400 MHz, CDCl₃) δ 9.08 (s, 1H), 7.42-7.23 (m., 8H), 7.13-7.06 (m, 2H), 5.72 (ddd, J=16.8, 10.4, 6.2 Hz, 1H), 5.43 (d, J=14.8 Hz, 1H), 5.28-5.09 (m, 2H), 4.62-4.56 (m, 1H), 4.13 (d, J=14.8 Hz, 1H), 3.91 (d, J=15.4 Hz, 1H), 3.62 (dd, J=15.4, 6.1 Hz, 1H), 2.35 (s, 3H). ¹³C NMR (101 MHz, CDCl₃) δ 169.05, 167.27, 162.40, 157.62, 145.00, 136.67, 132.34, 129.25, 129.25, 128,79, 128,79, 128.42, 128.42, 127.83, 127,44, 127.44, 126.68, 119.20, 109.72, 67.05, 52.89, 49.91, 25.94. HRMS (EST) calcd for C₂₃H₂₂N₄O [M+H]⁺: 371.1866, found: 371.1861.

Compound I-11: (S)-6-benzyl-2-(4-methyl-1-piperazinyl)-9-phenyl-8-vinyl-6,7,8,9-tetrahydro-5H-pyrimido[4,5-e]diazepine-5-one: white solid, 71 mg, yield 78%; MP: 187.3-188.1° C. 79% ee; Ee value is detected by chiral chromatographic column (mobile phase, n-hexane/2-propanol=70/30, detection temperature: 25° C., other parameters are the same as compound I-1, retention time: 10.481 min, 14.420 min), [α]_{D}²⁰=+21.2° (c=0.12, CHCl₃). H NMR (400 MHz, CDCl₃) δ 8.94 (s, 1H), 7.38-7.27, (m, 7H), 7.2.5-7.20 (m, 1H), 7.13-7.07 (m, 2H), 5.76 (ddd, J=16.9, 10.4, 6.4 Hz, 1H), 5.44 (d, J=14.9 Hz, 1H), 5.25-5.11 (m, 2H), 4.55-4.46 (m, 1H) 4.10 (d, J=14.9 Hz, 1H), 3.93 (d, J=15.2 Hz, 1H), 3.80-3.15 (m, 5H), 2.35-2.10 (s, 7H). ¹³C NMR (101 MHz, CDCl₃) δ 167.75, 164.36, 160.62, 158.38, 145.60, 137.26, 133.04, 128.95, 128.95, 128.67, 128.67, 128.31, 128.31, 127.86, 127.86, 127.56, 126.41, 118.78, 102.32, 66.57, 54.81, 54.81, 52.65, 50,21, 46.17, 43.38, 43.38. HRMS (ESI) calcd for C₂₇H₃₀N₆O [M+H]⁺: 455.2554, found: 455.2562.

Compound I-12: (S)6-benzyl-9-phenyl-2-(1-tetrahydropyrrolyl)-8-vinyl-6,7,8,9-tetrahydro-5H-pyrimido[4,5-e][1,4]diazepin-5-one: white solid, 70 mg, yield 82%; MP: 153.8-154.3° C. 90% ee; Ee value is detected by chiral chromatographic column (other parameters are the same as compound I-1, retention time: 11.038 min, 15.181 min), [α]_{D}²⁰ =−20.4° (c=0.16, CHCl₃). ¹H NMR (400 MHz, CDCl₃) δ 8.98 (s, 1H), 7.40-7.29 (m, 7H), 7.27-7.22 (m, 1H), 7.15-7.09 (m, 2H), 5.78 (ddd, J=16.9, 10.3, 6.4 Hz, 1H), 5.46 (d, J=14.8 Hz, 1H), 5.27-5.14 (m, 2H), 4.56-4.47 (m, 1H), 4.13 (d, J=14.9 Hz, 1H), 3.96 (d, J=15.2 Hz, 1H), 3.69-3.36 (m, 9H). ¹³C NMR (101 MHz, CDCl₃) δ 166.57, 163.19, 159.59, 157.29, 144.45, 136.15, 131.86, 127.94, 127.94, 127.61, 127.61, 127.25, 127.25, 126.77, 126.52, 125.48, 117.73, 101.58, 65.59, 65.53, 51.64, 49.14, 42.89, 42.89, 28.62, 28.62. HRMS (ESI) calcd. for C₂₆H₂₇N₅O [M+H]⁺: 426.2288, found: 426.2272.

Compound I-13: (R)-6-benzyl-2,9-diphenyl-8-vinyl-6,7,8,9-tetrahydro-5H-pyrimido[4,5-e][1,4]diazepine-5-one: white solid, 53 mg, yield 62%; MP: 212.7-213.6° C. 76% ee; Ee value is detected by a chiral chromatography column (other parameters are the same as compound I-6, retention time: 25.880 min, 28.153 min [α]_{D}²⁰=−25.4° (c=0.12, CHCl₃), ¹H NMR (400 MHz, CDCl₃) δ 9.29 (s, 1H), 8.10-7.96 (m, 2H), 7.50-7.43 (m, 2H), 7.43-7.30 (m, 9H), 7.25-7.18 (m, 2H), 5.80 (ddd, J=16.8, 10.4, 6.2 Hz, 1H), 5.49 (d, J=14.8 Hz, 1H), 5.35-5.20 (m, 4.68 (td, J=6.1, 1.2 Hz, 1H), 4.20 (d, J=14.9 Hz, 1H), 4.02 (d, J=15.4 Hz, 1H), 3.70 (dd, J=15.5, 6.0 Hz, 1H), ¹³C, NMR (101 MHz, CDCl₃) δ 167.19, 164.35, 163.03, 157.71, 145.05, 137.08, 136.70, 132.42, 130.90, 129.35, 129.35, 128.83, 128.83, 128.47, 128.47, 128.47, 128.29, 128.29, 127.86, 127.74, 127.74, 126.93, 119.25, 119.25, 110.05, 66.96, 52.98, 49.99. HRMS (ESI) calcd for C₂₈H₂₄N₄O [M+H]⁺: 433.2023, found: 433.2022.

Compound I-14: (S)-6-benzyl-2-(1-naphthyl)-9-phenyl-8-vinyl-6,7,8,9-tetrahydro-5H-pyrimido[4,5-e][1,4]diazepin-5-one: white solid, 84 mg, yield 88%; MP: 227.2-227.4° C. 86% ee; Ee value is detected by chiral chromatographic column (other parameters are the same as compound I-3, retention time: 21.740 min, 25.554 min), [α]_{D}²⁰=+24.4° (c=0.15, CHCl₃). ¹H NMR (400 MHz, CDCl₃) δ 9.35 (s, 1H), 8.56-8.46 (m, 1H), 8.23 (dd, J=7.4, 1.3 Hz, 1H), 7.85 (d, J=8.1 Hz, 1H), 7.76 (d, J=8.3, H), 7.50-7.40 (m, 3H), 7.40-7.27 (m, 7H), 7.24-7.19 (m, 2H), 7.18-7.10 (m, 1H), 5.81 (ddd, J=16.9, 10.3, 6.5 Hz, 1H), 5.51 (d, J=14.7 Hz, 1H), 5.33-5.17 (m, 2H), 4.70-4.51 (m, 1H), 4.17 (d, J=14.8 Hz, 1.14), 4.04 (d, J=15.4 Hz, 1H), 3.67 (dd, J=15.4, 5.8 Hz, 1H), ¹H NMR (101 MHz, CDCl₃) δ 167.24, 166.78, 162.59, 157.94, 145.21, 136.69, 134.11, 134.03, 132.44, 131.37, 131.18, 130.47, 129.67, 129.67, 128.86, 128.86, 128.47, 128.47, 128.22, 128.12, 128.12, 127.89, 127.18, 126.42, 126.31, 125.45, 125.00, 119.42, 109.66, 67.42, 52.97, 49.94, HRMS (ESI) calcd for C₃₂H₂₆N₄O [M+H]⁺: 483.2179, found: 483.2174.

Compound I-15: (S)-6-benzyl-9-phenyl-2-(3,4,5-trimethoxypheny)-8-vinyl-6,7,8,9-tetrahydro-5H-pyrimido [4,5-e][1,4]diazepin-5-one: white solid, 85 mg, 82% yield; MP: 115.0-115.4° C. 87% ee; Ee value is detected by chiral chromatographic column (mobile phase, n-hexane/2-propanol=65/35, other parameters are the same as compound I-3, retention time: 14.863 min, 17.718 min), [α]_{D}²⁰=+31.3° (c=0.20, CHCl₃). ¹H NMR (400 MHz, CDCl₃) δ 9.24 (s, 1H), 7.43 (m, 2H), 7.39-7.28 (m, 8H), 7.24-7.17 (m, 2H), 5.79 (ddd, J=16.8, 10.3, 6.3 Hz, 1H), 5.47 (d, J=14.7 Hz, 1H), 5.34-5.15 (in, 2H), 4.65 (t, J=5.9 Hz, 1H), 4.17 (d, J=14.8 Hz, H) 4.01 (d, J=15.4 Hz, 1H), 3.84 (s, 3H), 3.73 (s, 6H), 3.71-3.64 (m, 1H). ¹³C NMR (101 MHz, CDCl₃) δ 167.14, 163.54, 163.03, 157.53, 152.91, 152.91, 145.31, 140.49, 136.67, 132.42, 132.22, 129.31, 129.31, 128.82, 128.82, 128.47, 128.47, 128.23, 128.23, 127.86, 126.71, 119.30, 109.46, 105.32, 105.32, 66.86, 60.85, 55.93, 55.93, 53.00, 49.99. HRMS (ESI) calcd. for C₃₁H₃₀N₄O₄ [M+H]⁺: 523.2340, found: 523.2326.

Compound I-16: (R)-6-benzyl-2-(2-furyl)-9-phenyl-8-vinyl-6,7,8,9-tetrahydro-5H-pyrimido[4,5-e][1,4]diazepin-5-one: white solid, 71 mg, yield 84%; MP: 178.5-178.6° C. 92% ee; Ee value is detected by chiral chromatographic column (other parameters are the same as compound I-8, retention time: 26.611 min, 29.809 min), [α]_{D}²⁰=−42.3 0 (c=0.20, CHCl₃). ¹H NMR (400 MHz, CDCl₃)δ 9.25 (s, 1H), 7.53 (m, 1H), 7.44 (m, 2H), 7.41-7.31 (m, 6H), 7.22-7.15 (m, 2H), 6.71 (dd, J=3.4, 0.9 Hz, 1H), 6.41 (dd, J=3.5, 1.7 Hz, 1H), 5.78 (ddd, J=16.8, 10.4, 6.2 Hz, 1H), 5.46 (d, J=14.8 Hz, 1H), 5.31-5.17 (m, 4.70-4.61 (m, 1H), 4.19 (d, J=14.8 Hz, 1H), 4.00 (d, J=15.4 Hz, 1H), 3.69 (dd, J=15.4, 5.9 Hz, 1H). ¹³C NMR (101 MHz, CDCl₃) δ 166.90, 163.10, 157.71, 157.34, 151.87, 145.20, 144.80, 136.63, 132.34, 129.25, 129.25, 128.82, 128.82, 128.49, 128.49, 127.87, 127.64, 127.64, 126.91, 119.31, 114.34, 112.04, 109.95, 66.96, 52.99, 49.91. HRMS (ESI) calcd for C₂₆H₂₂N₄O₂ [M+H]⁺: 423.1816, found: 423.1814.

Compound I-17: (S)-6-benzyl-2-(4-methoxy-3-pyridyl)-9-phenyl-8-vinyl-6,7,8,9-tetrahydro-5H-pyrimido[4,5-e][1,4]diazepin-5-one: white solid, 74 mg, yield 80%; MP: 158.5-159.3° C. 85% ee; Ee value is detected by chiral chromatographic column (mobile phase, n-hexane/2-propanol=80/20, other parameters are the same as compound I-3, retention time: 10.868 min, 13.600 min), [α]_{D}²⁰=+23.4° (c=0.12, CHCl₃), ¹H NMR (400 MHz, CDCl₃) δ 9.22 (s, 1H), 8.74 (d, J=2.3 Hz, 1H), 8.18 (dd, J=8.7, 2.4 Hz, 1H), 7.43 (t, J=7.7 Hz, 2H), 7.38-7.27 (m, 6H), 7.21-7.10 (m, 2H), 6.66 (d, J=8.7 Hz, 1H), 5.77 (ddd, J=16.8, 10.4, 6.3 Hz, 1H), 5.46 (d, 14.8 Hz, 1H), 5.36-5.12 (m, 2H), 4.73-4.56 (m, 1H), 4.16 (d, J=14.8 Hz, 1H), 3.99 (d, J=15.4 Hz, 1H), 3.93 (s, 3H), 3.67 (dd, J=15.4, 6.0 Hz, 1H). ¹³C NMR (101 MHz , CDCl₃) δ 167.12, 165.77, 162.98, 157.63, 148.50, 144.91, 138.59, 136.65, 132.33, 129.45, 129.45, 128.82, 128.82, 128.45, 128.45, 127.86, 127.65, 127.65, 127.13, 126.46, 119.32, 110.32, 109.90, 67.07, 53.75, 52.96, 49.95, 29.72. HRMS (ESI) calcd for C₂₈I₂₅N₅O₂ [M+H]⁺: 464.2081, found: 464.2072.

Compound I18: (R)-6-benzyl-9-phenyl-2-anilino-8-vinyl-6,7,8,9-tetrahydro-5H-pyrimido[4,5-e][1,4]diazepine-5-one: white solid, 76 mg, yield 86%; MP: 232.1-232.3° C. 88% ee; Ee value is detected by chiral chromatographic column (mobile phase, n-hexane/2-propanol=70/30, other parameters are the same as compound I-1, retention time: 7.127 min, 12.460 min), [α]_{D}²⁰=−36.4° (c=0.10, CHCl₃). ¹H NMR (400 MHz, CDCl₃) δ 8.99 (s, 1H), 7.52-7.46 (m, 2H), 7.4-7.38 (m, 1H), 7.38-7.28 (m, 5H), 7.22-7.10 (m, 3H), 7.01-6.89 (m, 4H), 6.86-6.79 (m, 1H), 5.81 (ddd, J=17.0, 10.3, 6.6 Hz, 1H), 5.48 (d, J=14.8 Hz, 1H), 5.30-5.07 (m, 2H), 4.53 (t, J=6.2 Hz, 1H), 4.10 (d, J=14.8 Hz, 1H), 4.00 (d, J=15.3 Hz, 1H), 3.63 (dd, J=15.3, 5.8 Hz, 1H), ¹³C NMR (101 MHz, CDCl₃) δ 167.38, 164.09, 159.04, 159.01, 145.72, 139.26, 137.09, 132.56, 129.85, 129.85, 128.75, 128.75, 128.50, 128.50, 128.46, 128.46, 128.39, 128.39, 127.69, 127.05, 121.86, 119.18, 118.26, 118.26, 103.79, 67.13, 52.86, 50.09. HRMS (ESI) calcd for C₂₄H₂₅N₅O [M+H]⁺: 448.2132, found: 448.2120.

### Embodiment 4

According to the aforementioned substrate synthesis method, Embodiment 3, and the reaction conditions for synthesizing compound I1, specific compounds I19 to I-38 are prepared, and the results are shown in Table 2:

TABLE 2
Yields and enantiomeric excess values of compounds I-19 to I-38

| Compound | R¹ | R² | R³ | R⁴ | Yield (%) | ee value (%) |
|---|---|---|---|---|---|---|
| I-19 | H | Br— | 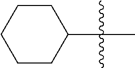 [SMILES: *C1CCCCC1] | HC═C—CH₂₋ | 56 | 88(R) |
| I-20 | —Si(CH₃)₃ | —CF₃ | Boc— | H | 76 | 82(R) |
| I-21 | Cl— | —NHCH₃ | ⁱPr— | Bz— | 64 | 89(R) |
| I-22 | CF₃CO— | 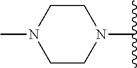 [SMILES: *N1CCN(C)CC1] | —CH₂CH═CH2 | 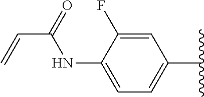 [SMILES: *c1ccc(NC(=O)C=C)c(F)c1] | 70 | 84(R) |
| I-23 | PhSO₂— | —Si(Ph)₃ | Troc— | Boc | 80 | 78(R) |
| I-24 | 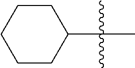 [SMILES: *C1CCCCC1] | 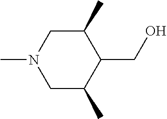 | 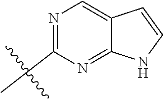 [SMILES: *c1ncc2cc[nH]c2n1] | 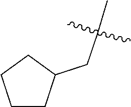 [SMILES: *CC1CCCC1] | 85 | 75(R) |
| I-25 | 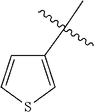 [SMILES: *c1ccsc1] | Bz— | H | 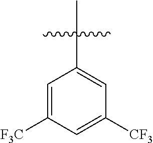 [SMILES: *c1cc(C(F)(F)F)cc(C(F)(F)F)c1] | 62 | 84(R) |
| I-26 | —NHCOCH₃ | PhCH₂O— | Bz— | Ts— | 80 | 87(R) |
| I-27 | —N(ⁱPr)₂ | MeSO₂— | Fmoc— | 2-THP— | 73 | 88(R) |
| I-28 | —OH | 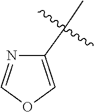 [SMILES: *c1cocn1] | Ts— | 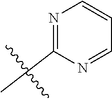 [SMILES: *c1ncccn1] | 83 | 91(R) |
| I-29 | —CN | —OH | 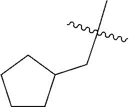 [SMILES: *CC1CCCC1] | 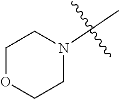 [SMILES: *N1CCOCC1] | 46 | 68(R) |
| I-30 | —COOH | —CN | 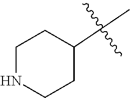 [SMILES: *C1CCNCC1] | 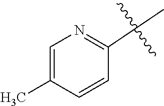 [SMILES: *c1ccc(C)cn1] | 52 | 70(R) |
| I-31 | —CF₃ | 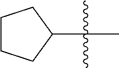 [SMILES: *C1CCCC1] | HC═C—CH₂₋ | 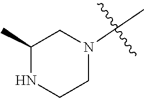 [SMILES: *N1CCN[C@@H](C)C1] | 65 | 78(R) |
| I-32 | —NH₂ | Et— | 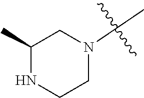 [SMILES: *N1CCN[C@@H](C)C1] | 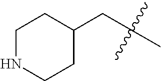 [SMILES: *CC1CCNCC1] | 72 | 74(R) |
| I-33 | —Bn | —COOH | 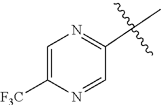 [SMILES: *c1cnc(C(F)(F)F)cn1] | 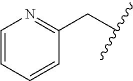 [SMILES: *Cc1ccccn1] | 60 | 82(R) |
| I-34 | —Si(Ph)₃ | —NHCOCH₃ | 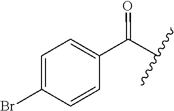 [SMILES: *C(=O)c1ccc(Br)cc1] | 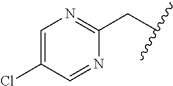 [SMILES: *Cc1ncc(Cl)cn1] | 75 | 74(R) |
| I-35 | 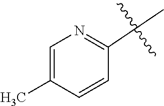 [SMILES: *c1ccc(C)cn1] | —NH₂ | Bn— | —CH₂CH═CH₂ | 62 | 80(R) |
| I-36 | —Si(Et)₃ | —N(Et)₂ | 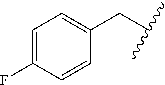 [SMILES: *Cc1ccc(F)cc1] | 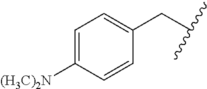 | 76 | 77(R) |
| I-37 | 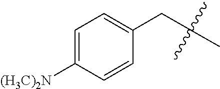 | Bn— | 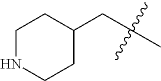 [SMILES: *CC1CCNCC1] | Ts— | 80 | 75(R) |
| I-38 | —NHEt | Ph— | 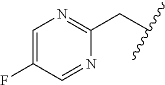 [SMILES: *Cc1ncc(F)cn1] | 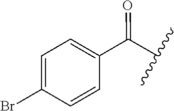 [SMILES: *C(=O)c1ccc(Br)cc1] | 73 | 81(R) |

### Embodiment 5: Evaluation of Antidepressant Activity of Pyrimidine-Fused Diazepinone Derivatives

### (1) Cell Culture and Establishment of Depression Model

PC12 cells are purchased from Procell Life Science & Technology Co., Ltd. (Wuhan, China). All cell culture reagents are purchased from Life Technologies (Grand Island, Nebraska, USA). Cells are cultured in Dulbecco's modified Eagle's medium (Gibco, USA) including 10% fetal calf serum (Gibco, USA) at 37° C. in an incubator including 5% CO₂.

Corticosterone (CORT)-induced PC12 cells are usually used to establish the depression model in vitro. The PC12 cells are treated with 600 μM corticosterone for 24 hours, at such corticosterone concentration, cell viability is reduced to 60% and can be used for subsequent vitro experiments.

### (2) Cell Viability Measurement

Cell viability is measured by using MTT [3-(4,5-dimethyl-2-thiazolyl)-2,5-diphenyl-2H-tetrazolium bromide]. Detailed steps include: inoculating 1×10⁵ cells into each well of a 96-well plate, after culturing for 24 hours for stabilization, adding 600 μM corticosterone, treating the cells for 24 hours, then removing culture medium including the corticosterone, and adding culture medium including drugs (e.g., compounds of the present disclosure/fluoxetine) at different concentrations for treating the cells for 12, 24, and 48 hours respectively, then adding MTT and incubating the cells at 37° C. for 4 hours, and measuring an absorbance of the cells in a microplate reader (BIO-RAD, USA) at 570 nm, the results are shown in FIG. 2 and Table 3. Table 3 shows the cell viability results of 24 h culture when a drug concentration is 0.625 uM.

### (3) Western Blotting

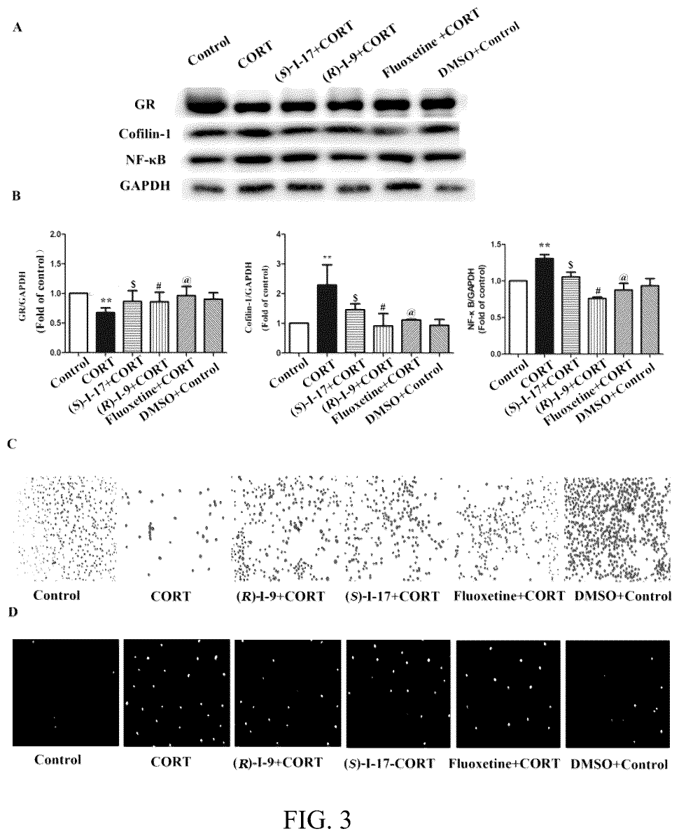

PC12 cells are lysed in RIPA lysis solution including 1% phenylmethylsulfonyl fluoride (PMSF) on ice for 30 minutes, the supernatant solution is collected and centrifuged, and the protein concentration of the supernatant solution is measured by a BCA assay kit (Beyotime, Shanghai). Protein samples are loaded onto 10% SDS-polyacrylamide gel electrophoresis and then further transferred to PVDF membrane (Millipore, MA, USA). The PVDF membrane is blocked with 5% non-fat milk and then incubated with a single primary antibody at 4° C. overnight. After washing with Tris-buffered saline containing Tween-20 (0.1%) (TBST), the PVDF membrane is incubated with secondary antibodies at room temperature. The PVDF membrane is analyzed by ECL reagents (Beyotime, Shanghai) and a BIO-RAD ChemiDoc XRS system. Protein band intensity is performed by Image J software (The National Institutes of Health, Bethesda, MD, USA). The results are shown in FIG. 3.

### (4) Hoechst and PI Staining

PC12 cell apoptosis is detected by Hoechst and PI staining. Hoechst can bind to cell nucleus of living cells, while PI (propidium iodide) can bind to the nucleuses of dead cells. The PC12 cells are plated in 12-well plates, and after being treated with drugs for 48 hours, the PC12 cells are stained with the nuclear dye Hoechst. The PC12 cells are washed twice with PBS and continued to be incubated with 10 mg/L Hoechst in the incubator for 20 min, For PI staining, the PC12 cells are incubated with a working solution including PI (final concentration 1 μg/mL) at room temperature for 10 min at 4° C. The PC12 cells are observed and photographed by a fluorescence microscope (Zeiss, Germany), and the results are shown in FIGS. 2 and 3. Blue fluorescence (white background) represents living cells, and red fluorescence (black background) represents dead cells.

### (5) Result Analysis:

TABLE 3
Measurement results of antidepressant activity of the pyrimidine-fused
diazepinone derivatives

| | Cell viability | | Cell viability | | Cell viability |
|---|---|---|---|---|---|
| Compound | (%)^{a} | Compound | (%)^{a} | Compound | (%)ª |
| (±)-I-1 | 88.7 ± 3.2 | (±)-I-10 | 89.3 ± 5.1 | (±)-I-22 | 78.3 ± 3.4 |
| (R)-I-1 | 82.8 ± 2.5 | (R)-I-10 | 77.0 ± 1.4 | (R)-I-22 | 72.9 ± 2.5 |
| (S)-I-1 | 89.3 ± 1.4 | (S)-I-10 | 97.8 ± 3.4 | (S)-I-22 | 84.1 ± 1.2 |
| (±)-I-2 | 74.2 ± 2.9 | (±)-I-11 | 95.6 ± 1.6 | (±)-I-23 | 84.2 ± 3.4 |
| (R)-I-2 | 71.2 ± 1,9 | (R)-I-11 | 82.2 ± 2.2 | (R)-I-23 | 78.9 ± 4.5 |
| (S)-I-2 | 92.6 ± 4.8 | (S)-I-11 | 98.5 ± 6.3 | (S)-I-23 | 92.0 ± 3.7 |
| (±)-I-4 | 82.6 ± 1.9 | (±)-I-12 | 93.8 ± 1.3 | (S)-I-27 | 85.6 ± 4.5 |
| (R)-I-4 | 74.2 ± 1.8 | (R)-I-12 | 87.4 ± 2.5 | (R)-I-28 | 81.2 ± 2.4 |
| (S)-I-4 | 95.3 ± 3.4 | (S)-I-12 | 98.6 ± 3.1 | (R)-I-29 | 74.9 ± 3.3 |
| (±)-I-5 | 82.6 ± 2.3 | (±)-I-13 | 84.2 ± 3.6 | (±)-I-30 | 87.9 ± 5.2 |
| (R)-I-5 | 78.8 ± 3.1 | (R)-I-13 | 81.9 ± 4.5 | (S)-I-31 | 84.4 ± 3.2 |
| (S)-I-5 | 89.4 ± 2.9 | (S)-I-13 | 95.7 ± 3.3 | (R)-I-32 | 88.6 ± 5.6 |
| (±)-I-7 | 86.9 ± 2.1 | (±)-I-14 | 83.9 ± 2.8 | (±)-I-33 | 75.7 ± 3.0 |
| (R)-I-7 | 71.6 ± 2.8 | (R)-I-14 | 73.2 ± 3.2 | (S)-I-34 | 89.2 ± 4.1 |
| (S)-I-7 | 90.0 ± 1.4 | (S)-I-14 | 88.1 ± 4.7 | (R)-I-35 | 88.3 ± 6.2 |
| (±)-I-8 | 85.6 ± 4.3 | (±)-I-17 | 83.5 ± 2.6 | (R)-I-36 | 79.4 ± 4.5 |
| (R)-I-8 | 82.0 ± 2.0 | (R)-I-17 | 71.3 ± 4.3 | (R)-I-37 | 86.5 ± 3.4 |
| (S)-I-8 | 88.6 ± 1.7 | (S)-I-17 | 96.9 ± 2.7 | (S)-I-38 | 69.6 ± 5.3 |
| (±)-I-9 | 92.8 ± 3.2 | (±)-I-18 | 88.4 ± 5.0 | 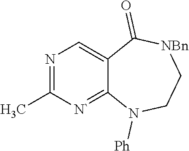 | 63.8 ± 2.3 |
| (R)-I-9 | 85.4 ± 2.9 | (R)-I-18 | 75.2 ± 4.4 | | |
| (S)-I-9 | 98.5 ± 4.2 | (S)-I-18 | 90.0 ± 0.8 | 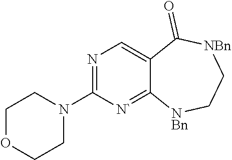 | 65.4 ± 1.2 |
| CORT^{b} | 60.5 ± 3.6 | fluoxetine^{c} | 76.8 ± 6.4 | | |

Note: a uses a concentration of 0.625 uM, and the value is an average value of three independent experiments; b is corticosterone; c is a positive control drug.

Cell survival rates at different drug concentrations (e.g., 20 uM, 10 uM, 5 uM, 2.5 uM, 1.25 uM and 0.625 uM) are measured by a MIT method at different times (12 h, 24 h and 48 h) according to the present disclosure, and the measurement results are shown in FIG. 2. Normal cells are taken as a blank control (control), corticosterone treated group (CORT) is taken as a control group, and DMSO+control are taken as solvent control group. A in FIG. 2 shows the results of drug effects at different concentrations after 12 hours of culture; B in FIG. 2 shows the results of drug effects at different culture times when the drug concentration is 0.625 uM. It can be seen from the figure that, corticosterone reduces the survival rate of PC12 cells to about 60%, and adding the compound (S)-I11 of the present disclosure and the positive drug fluoxetine can effectively reduce the death rate of cells caused by CORT, and the effect of the compound of the present disclosure is more significant than that of the positive drug fluoxetine, making the cells tend to normal cells. In addition, it can be seen from B of FIG. 2 that, the positive drug fluoxetine takes 48 hours to exert its effect, while the compound (S)-I-11 of the present disclosure shows excellent effect in 12 hours (as shown in A and B of FIG. 2).

The results of Hoechst and PI methods show that, the corticosterone causes PC12 cell death, which is manifested by a decrease in blue fluorescence number and an increase in red fluorescence number, while the compound (S)-I-11 has a similar effect to fluoxetine and can reduce cell death, which is manifested by an increase in blue fluorescence number and a decrease in red fluorescence number (as shown in C and D of FIG. 2, the concentration used is 1.0 uM).

The pathogenesis of MDD is related to HPA axis hyperactivity, synaptic remodeling, and inflammation. GR, cofilin-1, and NF-κB play key roles in the pathogenesis of MDD. The results show that, compounds (S)-I-17 and (R)-I-9 in the embodiments exhibit the same effects as fluoxetine, and can promote the expression of GR during depression and inhibit the overexpression of cofilin- and NF-κB proteins (as shown in A and B of FIG. 3); and compounds (S)-I-17 and (R)-I-9 can reverse the massive apoptosis of the PC12 cells caused by corticosterone (as shown in C and D of FIG. 3). These results further prove that compounds (S)-I-17 and (R)I-9 have good antidepressant activity in vitro.

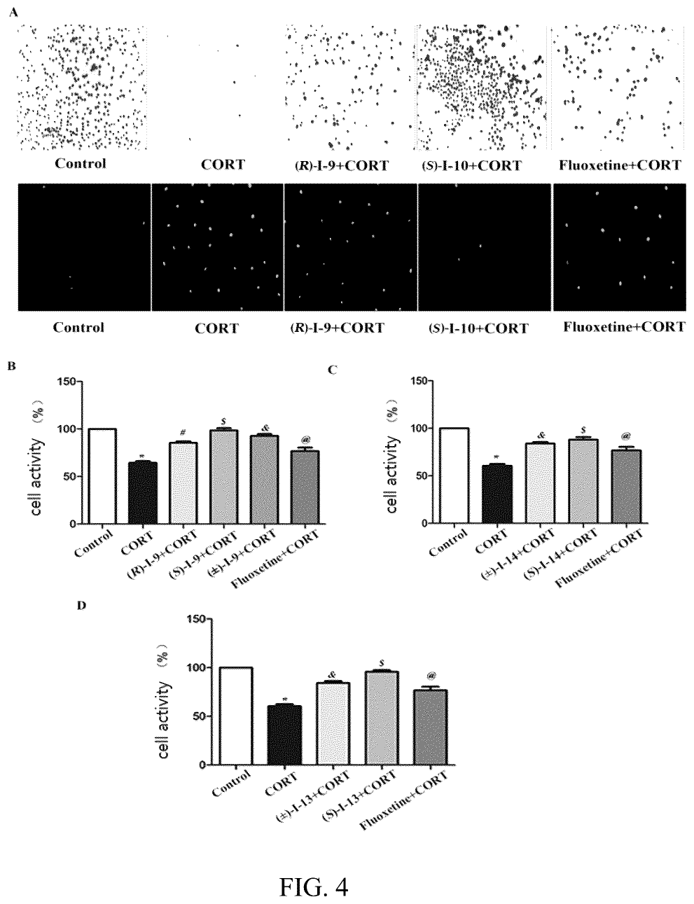

In addition, it can be seen from FIG. 4 that, compound (S)-I-10 yields a higher number of blue fluorescence than (R)-I-9 and a lower number of red fluorescence than (R)-I-9 (as shown in A in FIG. 4); and compared with (R)-I-9, (S)-I-9 can better increase the survival rate of the PC12 cells (as shown in B of FIG. 4). (S)-I-14 and (5)-I-1 can increase the survival rate better than their corresponding racemates (as shown in C and D of FIG. 4). These results indicate that, the pyrimidine-fused diazepin.one compounds with the (S)-configuration may have better antidepressant effects than the (R)-configuration compounds and their corresponding racemates.

Combined with Table 3 and the accompanying drawings, it is shown that the pyrimidine-fused diazepinone derivatives of the present disclosure have good antidepressant effects, and even the antidepressant activity of some compounds is better than that of the positive control drug fluoxetine; Moreover, the antidepressant activity of the compounds introduced into the vinyl moiety in the present disclosure is significantly better than that of the compounds without the vinyl moiety.

### Embodiment 6: Experiment on Mouse Depression Model of the Pyrimidine-Fused Diazepinone Derivatives

A chronic depression model is induced in mice by intraperitoneal injection of reserpine 0.4 mg/kg for 14 days. A tail suspension test (TST), a forced swimming test (FST), a sucrose preference test (SWP), and an open field test (OFT) are used as criteria to evaluate degree of depression. After the chronic depression model is established, fluoxetine is used as a positive control drug, and the compound of the present disclosure is used for treatment for 14 days, to determine the antidepressant effect of the compound of the present disclosure; a single dose is used to determine the onset time of the compound.

### Experimental Steps

Tail suspension test (TST): fix approximately 1 cm from an end of a tail of a mouse with tape and hang it. Use a camera to record the mouse's activities within 6 minutes, and statistically analyze the mouse's immobility time within last 4 minutes.

Forced swimming test (FST) place the mouse alone in a glass jar (height 20 cm, diameter 14 cm) with a water depth of 15 cm, water temperature (25±1)° C., let the mouse swim for 6 minutes, and observe and record the immobility time of the mouse in the water within last 4 minutes.

Sucrose preference test (SWP): Rodents are born with a strong preference for sweets. When the mouse is provided with two drinking devices with free choices of sucrose solution and ordinary water, the 24-hour sugar water consumption is calculated. Sucrose preference ratio (%)=sugar water consumption/(sugar water consumption+pure water consumption)×100%.

Open Field Experiment (OFT): the experiment is carried out in a quiet environment. The mouse is placed in a center of a bottom of a 40×40 cm box, and a video behavior analysis system is used to observe and record the activities of the mouse in the open field test box for 15 minutes.

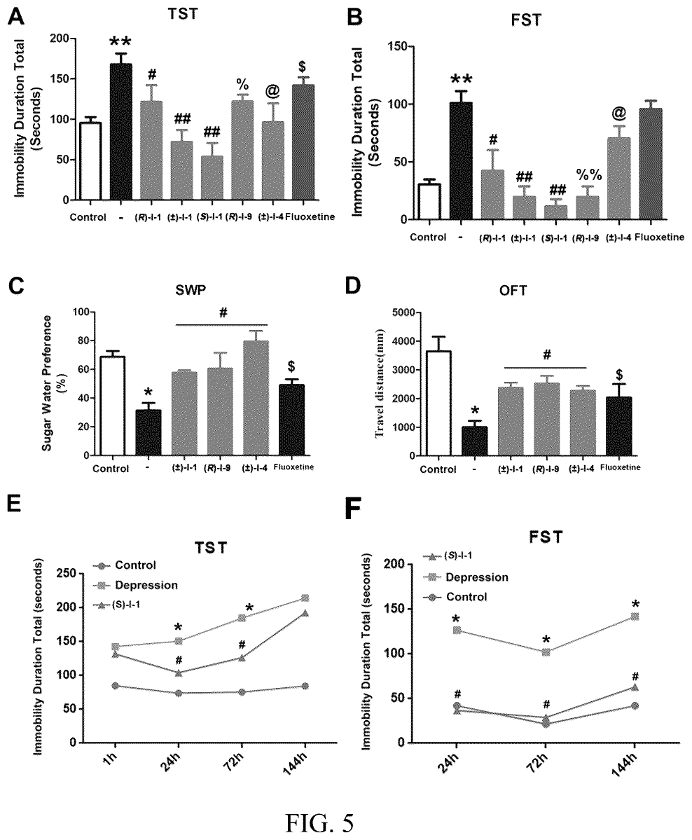

FIG. 5 illustrates the experimental results of the derivatives of the present disclosure on reserpine-induced depression model mice. A is the mouse tail suspension test; B is the mouse forced swimming test; C is the mouse sucrose preference test; D is the mouse open field test; F is the single dose mouse tail suspension test; F is single dose forced swimming experiment

Normal mice without MDD are used as a blank control (control), and mice with MDD without medication are used as a control group (expressed as “-” in A of FIG. 5 to D of FIG. 5; expressed as “depression” in E of FIG. 5 and F of FIG. 5).

The results indicate that: compared with the diseased control group, pyrimidine-fused diazepinone derivatives of the present disclosure can significantly reduce the immobility time of TST (as shown in A of FIG. 5) and the immobility time of EST (as shown B in FIG. 5) of the mice with MDD, can restore the mice's preference for sucrose (as shown C in FIG. 5), and can increase a moving distance of the mice in the central zone (as shown. D in FIG. 5), thus making them tend to normal mice, which is consistent with the influence trend of the positive drug fluoxetine, and the effect is more significant than the positive drug fluoxetine, which indicates that the compounds of the present disclosure has a better antidepressant effect than the positive drug fluoxetine.

Further, it can be seen from E of FIG. 5 and F of FIG. 5 that, the pyrimidine-fused diazepinone derivatives of the present disclosure can produce obvious antidepressant effects 24 hours after administration, which makes the depressed mice behave line normal mice (as shown in FIG. 5), and it is significantly better than the onset time of fluoxetine; and the effect lasts for 144 hours (as shown in F in FIG. 5), thereby indicating that the antidepressant effect of the compounds of the present disclosure is rapid and lasting.

Based on the above results, it s indicated that the pyrimidine-fused diazepinone derivatives of the present disclosure have excellent, rapid-acting antidepressant effects and long-lasting effects.

The above embodiments are preferred embodiments of the present disclosure, but the embodiments of the present disclosure are not limited to the above embodiments, any other changes, modifications, substitutions, combinations, simplifications made without departing from the spirit and principles of the present disclosure, should be equivalent substitutions, and are all included in the protection scope of the present disclosure.

What is claimed is:

1. Chiral or racemic pyrimidine-fused diazepinone derivatives or pharmaceutically acceptable salts thereof, wherein a structural formula of the pyrimidine-fused diazepinone derivatives is as shown in formula (I), a carbon atom marked with * is a chiral carbon atom with a configuration of R, S, or R/S, and the pyrimidine-fused diazepinone derivatives is a levorotatory form, a dextrorotatory form, or a racemic form; 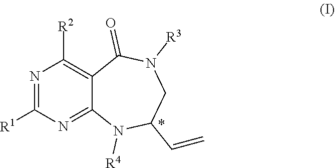
wherein R¹ and R² are independently selected from hydrogen, halogen atom, hydroxyl group, carboxyl group, cyano group, nitro group, C1-C20 linear or branched alkyl group, C1-C20 fluoroalkyl group, C2-C20 alkenyl group, C2-C20 alkynyl group, C1-C20 alkoxy group, C3-C20 cycloalkyl group, C1-C20 amide group, C2-C20 ketone carbonyl group, C1-C20 sulfonyl group, C1-C9 alkyl silyl group, phenyl silyl group, amino group, C1-C20 N-alkyl substituted amino group, C1-C20 N,N-dialkyl substituted amino group, substituted or unsubstituted C3-C20 heterocyclic group or heterocyclic aryl group containing one or more of N, O, and S, substituted or unsubstituted aryl group, substituted or unsubstituted aryl methyl group;
R³ and R⁴ are independently selected from hydrogen, C1-C20 linear or branched alkyl, C3-C20 cycloalkyl, C3-C20 cycloalkyl methylene group, C3-C20 allyl, C3-C20 propargyl group, C1-C20 acyl group, C1-C20 sulfonyl group, substituted or unsubstituted C3-C20 heterocyclic group or heterocyclic aryl group containing one or more of N, O and S, substituted or unsubstituted heterocyclic methyl group or heterocyclic aryl methyl group, substituted or unsubstituted C1-C20 alkoxycarbonyl group, substituted or unsubstituted aryl acyl group, substituted aryl sulfonyl group, substituted or unsubstituted aryl group, substituted or unsubstituted aryl methyl group.

2. The chiral or racemic pyrimidine-fused diazepinone derivatives or pharmaceutically acceptable salts thereof according to claim 1, wherein: in R¹ and R², one or more hydrogen atoms of the C1-C20 linear or branched alkyl group, the C1-C20 fluoroalkyl group, the C2-C20 alkenyl group, the C2-C20 alkynyl group, the C1-C20 alkoxy group, the C3-C20 cycloalkyl group, the C1-C20 amide group, the C2-C20 ketone carbonyl group, the C1-C20 sulfonyl group, the C1-C9 alkyl silyl group, the phenyl silyl group ; the amino group, the C1-C20 N-alkyl substituted amino group, the C1-C20 substituted amino group; are substituted by fluorine atoms, chlorine atoms, bromine atoms, oxygen atoms, alkenyl groups, alkynyl groups, aryl groups, hydroxyl groups, amino groups, carbonyl groups, carboxyl groups, ester groups, cyano groups, methyl groups, ethyl groups, methoxy groups, nitro groups;
in R³ and R⁴, one or more hydrogen atoms of the C1-C20 linear or branched alkyl group, the C3-C20 cycloalkyl group, the C3-C20 cycloalkyl methylene group, the C3-C20 allyl group, the C3-C20 propargyl group, the C1-C20 acyl group, the C1-C20 sulfonyl group, are substituted by fluorine atoms, chlorine atoms, bromine atoms, oxygen atoms, alkenyl groups, alkynyl groups, aryl groups, hydroxyl groups, amino groups, carbonyl groups, carboxyl groups, ester groups, cyano groups, methyl groups, ethyl groups, methoxy groups, nitro groups.

3. The chiral or racemic pyrimidine-fused diazepinone derivatives or pharmaceutically acceptable salts thereof according to claim 1, wherein the structural formula is the formula (I) with a skeleton structure of the pyrimidine-fused diazepinone,
wherein R¹ and R² are independently selected from hydrogen, fluorine, chlorine, bromine, iodine, hydroxyl, carboxyl, cyano, nitro, methyl, ethyl, n-propyl, isopropyl, n-butyl, isobutyl ; tert-butyl, trifluoromethyl, benzyl, methoxy, ethoxy, n-propoxy, isopropoxy, n-butoxy, isobutoxy, tert-butoxy, benzyloxy group, amino group, C1-C20 amide group, trimethylsilyl group, triethylsilyl group, triphenylsilyl group, C3-C20 heterocyclic group or heterocyclic aryl group containing one or more of N, O, and S, C1-C20 N-alkyl substituted amino group, C1-C20 N,N-dialkyl substituted amino group, C2-C20 ketone carbonyl group, C1-C20 sulfonyl group, substituted or unsubstituted aryl group; a substituent in the substituted aryl group is one or more combinations of C1-C20 alkyl, halogen, or C1-C20 alkoxy group;
R³ and R⁴ are independently selected from hydrogen, C1-C20 linear or branched alkyl, C3-C20 cycloalkyl, C3-C20 cycloalkyl methylene group, C3-C20 heterocyclic group or heterocyclic aryl group containing one or more of N, O, and S, heterocyclic methylene group or heterocyclic aryl methylene group, allyl, propargyl, acetyl, benzoyl, C1-C20 sulfonyl group, tert-butoxycarbonyl group, fluorenyl methoxycarbonyl group, 2,2,2-trichloroethoxycarbonyl group, substituted or unsubstituted aryl group, substituted or unsubstituted aryl methylene group, substituted or unsubstituted benzenesulfonyl group; the substituted substituents are independently selected from one or more combinations of hydrogen, C1-C20 alkyl, C1-C20 fluoroalkyl, halogen, nitro, or C1-C20 alkoxy group.

4. The chiral or racemic pyrimidine-fused diazepinone derivatives or pharmaceutically acceptable salts thereof according to claim 1, wherein the structural formula is the formula (I) with a skeleton structure of the pyrimidine-fused diazepinone:
wherein R¹ and R² are independently selected from hydrogen, fluorine, chlorine, bromine, iodine, methyl, ethyl, n-propyl, isopropyl, n-butyl, isobutyl, tert-butyl, trifluoromethyl group, methoxy, ethoxy, n-propoxy, isopropoxy, n-butoxy, isobutoxy, tert-butoxy, hydroxyl, carboxyl, cyano, cyclopentyl, cyclohexyl, amino, methylamino, ethylamino, diethylamino, diisopropylamino, trimethylsilyl, triethylsilyl, triphenylsilyl, acetamido, acetyl, trifluoroacetyl group, phenyl, anilino group, benzoyl. 3,4,5-trimethoxyphenyl, benzyl, 4-dimethylaminobenzyl, benzyloxy, methanesulfonyl, phenylsulfonyl, naphthyl, morpholinyl, pyrrolyl, tetrahydropyrrolyl, 1-piperazinyl, 1-methylpiperazinyl, pyridyl, 4-methylpyridyl, methoxypyridyl, furyl, piperidinyl, 4-hydroxymethyl-3,5-dimethylpiperidinyl, thienyl, oxazolyl.

5. The chiral or racemic pyrimidine-fused diazepinone derivatives or pharmaceutically acceptable salts thereof according to claim 1, wherein the structural formula is the formula (I) with a skeleton structure of the pyrimidine-fused diazepinone:
wherein R³ and R⁴ are independently selected from hydrogen, methyl, ethyl, n-propyl, isopropyl, cyclohexyl, cyclopentylmethyl, allyl, propargyl, carbonyl, ethoxycarbonyl, tert-butoxycarbonyl, trichloroethoxyformyl, benzoyl, 4-bromobenzoyl, 9-fluorenylidenemethoxyformyl, 3-fluoro-4-(allylamido)phenyl, sulfonyl, tosyl, phenyl, 4-methoxyphenyl, 4-(trifluoromethyl)phenyl, 3, 5-bis(trifluoromethy 1)phenyl, benzyl, p-fluorobenzyl, 4-dimethylaminobenzyl, 4-methoxybenzyl, 2-tetrahydropyranyl, 4-(trifluoromethyl)benzyl, 3,5-bis(trifluoromethyl)benzyl, pyrimidyl, 4-fluoropyrimidinylmethyl, 4-chloropyrimidinylmethyl, pyrrolopyrimidinyl, morpholinyl, pyridyl, 3-methylpyridyl, pyridylmethyl, pyrazinyl, 4-trifluoro methyl pyrazinyl, piperazinyl, 3-methylpiperazinyl, piperidinyl, piperidylmethyl.

6. A preparation method of chiral or racemic pyrimidine-fused diazepinone derivatives in claim 1, the method comprising using pyrimidine allyl compound intermediates as raw materials, using iridium complexes formed by an interaction between iridium compounds and phosphoramidite ligands as catalysts, and obtaining the chiral or racemic pyrimidine-fused diazepinone derivatives by reactions under action of alkalis.

7. The preparation method according to claim 6, wherein: a structural formula of the pyrimidine allyl compound intermediates is as shown in a formula (S), 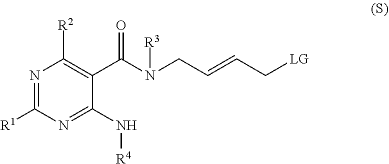
wherein LG is a leaving group, which is hydroxyl, chlorine, bromine, 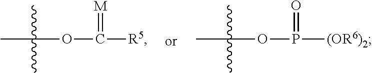
M is NH or O, R⁵ is a halogen-substituted or unsubstituted C1-C20 alkyl group, a halogen-substituted or unsubstituted C1-C20 alkoxy group; R⁶ is a C1 -C20 alkyl group, a substituted or unsubstituted aryl group.

8. The preparation method according to claim 7, wherein: the pyrimidine allyl compound intermediates are prepared by a method comprising following steps: generating 2-chloro-4-substituted amino-6-substituent-pyrimidine-5-carboxylic acid methyl ester compounds by a reaction between 2,4-dichloro-6-substituent-pyrimidine-5-carboxylic acid methyl ester compounds and amine compounds, introducing a group R¹ through a conventional nucleophilic substitution reaction or coupling reaction, generating carboxyl compounds by hydrolysis, and obtaining the compounds of the formula S by reacting with

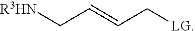

9. The preparation method according to claim 5, wherein the preparation method comprises following steps:
(1) obtaining compounds 3 through a nucleophilic substitution reaction by using 2,4-dichloro-5-pyrimidinecarboxylic acid ethyl ester compounds 1 and amine compounds 2 as starting materials, and using N,N-diisopropylethylamine as alkali;
(2) obtaining a variety of substituted compounds 4 by a conventional coupling reaction or the nucleophilic substitution reaction between the compounds 3 and boric acid compounds, amine compounds, or sodium alkoxide compounds, in the presence of anhydrous solvents and alkalis;
(3) obtaining compounds 5 by a hydrolysis reaction of compounds 4 in water and in the presence of the alkalis;
(4) obtaining pyrimidine allyl compound intermediates by the compounds 5 being condensed with compounds 6 under the action of 1-hydroxybenzotriazole and 1-ethyl-(3-dimethylaminopropyl) carbodiimide hydrochloride, which is used as substrates for a subsequent catalytic allyl amination reaction;
(5) obtaining the compounds shown in the formula (I) by an intramolecular allyl amination reaction produced by metallic iridium complexes formed by iridium compounds and phosphoramidite ligands catalyzing the substrates in organic solvent in the presence of organic or inorganic alkalis.

10. An application of a chiral or racemic pyrimidine-fused diazepinone derivatives or pharmaceutically acceptable salts thereof in claim 1 comprises an application in the preparation of drugs or their lead compounds for preventing or treating depressive disorder.
